(19) United States
(12) Patent Application Publication
(10) Pub. No.: US 2026/0130936 A1
(43) Pub. Date: May 14, 2026

(54) COMPOSITIONS COMPRISING AN ACTIVE INGREDIENT MIXTURE COMPRISING SILICON AND BIOTIN AND METHODS OF USE

(71) Applicant: Nutrition21, LLC, Saddle Brook, NJ (US)

(72) Inventors: James R. Komorowski, Trumbull, CT (US); Sara Perez Ojalvo, Saddle Brook, NJ (US)

(21) Appl. No.: 19/436,348

(22) Filed: Dec. 30, 2025

Related U.S. Application Data
(63) Continuation of application No. 18/841,884.
(60) Provisional application No. 63/314,794, filed on Feb. 28, 2022.

(51) Int. Cl. A61K 33/06; A61K 31/047; A61K 31/198; A61K 31/4188; A61P 17/16

(52) U.S. Cl. CPC A61K 33/06; A61K 31/047; A61K 31/198; A61K 31/4188; A61P 17/16

(57) ABSTRACT

Provided herein are compositions comprising an active ingredient mixture comprising silicon and biotin, compositions comprising an active ingredient mixture comprising inositol-stabilized arginine silicate complex (ASI) and magnesium biotinate, and compositions formulated to deliver silicon and biotin to a subject. Methods of using said compositions for improving the appearance of a subject's skin, hair, or nails; as well as methods for treating or preventing a condition in a subject are also provided herein.

### RELATED APPLICATIONS

This application claims the benefit of U.S. Provisional Application No. 63/314,794, filed Feb. 28, 2022, the contents of which are fully incorporated by reference herein.

### BACKGROUND

Skin, hair, and nails are key aspects of a person's physical appearance. As such, an improvement in the appearance of a person's skin, hair, or nails can provide an overall improvement in physical appearance, which is associated with greater happiness, self-confidence, and quality of life. However, whether due to aging, a lack of nutrients, ailments, or environmental factors, the appearance of skin, hair, and nails tends to diminish over time. Therefore, there is a strong desire for compositions and methods that improve the appearance of skin, hair, and nails.

### SUMMARY

In certain aspects, provided herein is a composition comprising an active ingredient mixture comprising silicon and biotin.

In further aspects, provided herein is a composition comprising an active ingredient mixture comprising inositol-stabilized arginine silicate complex (ASI) and magnesium biotinate.

In further aspects, provided herein is a composition formulated to deliver silicon and biotin to a subject.

In further aspects, provided herein is a method of improving the appearance of a subject's skin, hair, or nails comprising administering a composition comprising an active ingredient mixture comprising silicon and biotin to the subject.

In further aspects, provided herein is a method of treating or preventing a condition in a subject comprising administering a composition comprising an active ingredient mixture comprising silicon and biotin to the subject.

### BRIEF DESCRIPTION OF THE DRAWINGS

FIGS. 1-3 are histopathological images illustrating an effect of a composition of inositol-stabilized arginine silicate complex (ASI) and magnesium biotinate (MgB) on histopathological changes of skin tissue stained with hematoxylin and eosin (H&E, 100×; FIG. 1), showing relative skin thickness, Masson's trichrome (MT, 100×; FIG. 2), and Elastin (E, 400×; FIG. 3). In the histopathological images, the groups are shown as:
a: NC (normal control; normal histology of epidermis and dermis (arrow) and tightly arranged collagen fibers (star));
b: SC [shaved control; normal histology of epidermis and dermis (arrow) and tightly arranged collagen fibers (star)];
c: UVB (An increase in epidermis thickness (double arrow) and loosely arranged dermal collagen fibers (star);
d: ASI+Low Dose Magnesium Biotinate (MgB-L);
e: ASI+High Dose Magnesium Biotinate (MgB-H);
f: ASI+MgB-L+Magnesium Biotinate Cream (MgB-C); and
g: ASI+MgB-H+MgB-C.

ASI and MgB treatments alleviated UVB-induced skin damage features, particularly ASI+MgB-H+MgB-C, showing close to normal epidermal morphology (arrow) and close to normal arranged collagen fibers (star). Masson's trichrome staining shows collagen (arrow and stars). Elastin staining (FIG. 3) shows decreased collagen fibers (arrows) in UVB, and treatments alleviated the damages.

FIGS. 4-6 are graphs illustrating an effect of a composition of inositol-stabilized arginine silicate complex (ASI) and magnesium biotinate (MgB) on epidermal thickness (FIG. 4), microfolds (FIG. 5), and inflammatory cells (FIG. 6) in rats with UVB-induced photoaging. Each bar represents the mean and standard error of the mean. a-d: Values within the bars with different superscripts are significantly different (One-way ANOVA and Tukey's post-hoc test, p<0.05).

FIGS. 7-10 are immunohistochemical images illustrating an effect of administration of a composition of inositol-stabilized arginine silicate complex (ASI) and magnesium biotinate (MgB) on changes of skin tissue stained with immunohistochemical stain for the mammalian target of rapamycin (p-mTOR; FIG. 7), matrix metalloproteinase-1 (MMP-1; FIG. 8), interleukin 6 (IL-6; FIG. 9), and cyclooxygenase (COX-2; FIG. 10) in rats with UVB-induced photoaging. In the immunohistochemical images, the groups are shown as:
a: NC, normal control;
b: SC, shaved control;
c: UVB;
d: ASI+MgB-L;
e: ASI+MgB-H;
f: ASI+MgB-L+MgB-C; and
g: ASI+MgB-H+MgB-C.

FIGS. 11-14 are graphs illustrating an effect of administration of a composition of inositol-stabilized arginine silicate (ASI) and magnesium biotinate (MgB) on mTOR (FIG. 11), MMP-1 (FIG. 12), IL-6 (FIG. 13), and COX-2 (FIG. 14), the staining (0, 0%; 1, <25%; 2, 25-50%; 3, 51-75%; and 4, >75%) were scored. a-e: Values within the bars with different superscripts are significantly different (Kruskal-Wallis and Mann Whitney U test, p<0.05).

FIGS. 15-20 are graphs illustrating an effect of administration of a composition of inositol-stabilized arginine silicate (ASI) and magnesium biotinate (MgB) on acetyl COA carboxylase 1 (ACC-1; FIG. 15), acetyl COA carboxylase 2 (ACC-2; FIG. 16), pyruvate carboxylase (PC; FIG. 17), propionyl-CoA carboxylase (PCC; FIG. 18), 3-methylcrotonyl-CoA carboxylase (MCC; FIG. 19) protein levels in rats with UVB-induced photoaging. Data are expressed as a percent of the control value. Each bar represents the mean and standard error of the mean. Blots were repeated at least 3 times. Western blot analysis was performed with actin included ensuring equal protein loading (FIG. 20). a-f: Values within the bars with different superscripts are significantly different (One-way ANOVA and Tukey's post-hoc test, p<0.05).

FIGS. 21-28 are graphs illustrating an effect of administration of a composition of inositol-stabilized arginine silicate (ASI) and magnesium biotinate (MgB) on the mammalian target of rapamycin complex2 (p-mTORC2; FIG. 21), ribosomal protein S6 kinase beta-1 (p-p70S6K; FIG. 22), eukaryotic initiation factor 4E-binding protein 1 (p4E-BP1; FIG. 23), vascular endothelial growth factor (VEGF; FIG. 24), sirtuin 1 (SIRT1; FIG. 25), matrix metalloproteinase-1 (MMP-1; FIG. 26), and matrix metalloproteinase-3 (MMP-3; FIG. 27) protein levels in rats with UVB-induced photoaging. Data are expressed as a percent of the control value. Each bar represents the mean and standard error of the mean. Blots were repeated at least 3 times. Western blot analysis was performed with actin included ensuring equal protein loading (FIG. 28). a-f: Values within the bars with different superscripts are significantly different (One-way ANOVA and Tukey's post-hoc test, p<0.05).

FIGS. 29-34 are graphs illustrating an effect of administration of a composition of inositol-stabilized arginine silicate (ASI) and magnesium biotinate (MgB) on tumor necrosis factor-alpha (TNF-α; FIG. 29), nuclear factorkappa B (NF-κB; FIG. 30), interleukin 6 (IL-6; FIG. 31), interleukin 8 (IL-8; FIG. 32), and cyclooxygenase (COX-2; FIG. 33) protein levels in rats with UVB-induced photoaging. Data are expressed as a percent of the control value. Each bar represents the mean and standard error of the mean. Blots were repeated at least 3 times. Western blot analysis was performed with actin included ensuring equal protein loading (FIG. 34). The data are percentages of the control. a-e: Values within the bars with different superscripts are significantly different (One-way ANOVA and Tukey's post-hoc test, p<0.05).

FIGS. 35-39 are graphs illustrating an effect of administration of a composition of inositol-stabilized arginine silicate (ASI) and magnesium biotinate (MgB) on c-Jun N-terminal kinase (JNK; FIG. 35), p38 mitogen-activated protein kinase (MAPK; FIG. 36), AP1, activator protein 1 (c-jun; FIG. 37 and c-fos; FIG. 38) protein levels in rats with UVB-induced photoaging. Data are expressed as a percent of the control value. Each bar represents the mean and standard error of the mean. Blots were repeated at least 3 times. Western blot analysis was performed with actin included ensuring equal protein loading (FIG. 39). The data are percentages of the control. a-e: Values within the bars with different superscripts are significantly different (One-way ANOVA and Tukey's post-hoc test, p<0.05).

FIGS. 40-43 are graphs illustrating an effect of administration of a composition of inositol-stabilized arginine silicate (ASI) and magnesium biotinate (MgB) on the Bax (FIG. 40), Bcl-2 (FIG. 41) and caspase-3 (FIG. 42) levels in rats with UVB-induced photoaging. Data are expressed as a percent of the control value. Each bar represents the mean and standard error of the mean. Blots were repeated at least 3 times. Western blot analysis was performed with actin included ensuring equal protein loading (FIG. 43). The data are percentages of the control. a-e: Values within the bars with different superscripts are significantly different (One-way ANOVA and Tukey's post-hoc test. p<0.05).

FIG. 44 is a graph illustrating the effects of a composition according to exemplary embodiments of the invention on skin appearance and health in rats chronically exposed to ultraviolet (UV) radiation.

FIG. 45 is a graph illustrating the effects of a composition according to exemplary embodiments of the invention on the effect of the percentage of hair growth.

### DETAILED DESCRIPTION

In certain aspects, provided herein are compositions and methods that that improve the appearance of skin, hair, and nails. In certain embodiments, the methods described herein comprise administering a composition comprising an active ingredient mixture comprising silicon and biotin to a subject.

Silicon (Si) is the third most abundant trace mineral in the human body and is important for the synthesis of collagen, activating hydroxylation enzymes, and improving skin strength and elasticity. It has also been shown to improve hair brightness and to prevent hair loss.

The vitamin biotin has been shown to play a major role in the synthesis of protein including keratin, the fibrous protein that forms the main structural constituent of hair and nails. It is also a coenzyme for the mitochondrial carboxylases in hair roots. Biotin is also a vital cofactor for the five biotin-dependent carboxylases, including acetyl-CoA carboxylases 1 and 2 (ACC1 and ACC2), pyruvate carboxylase (PC), 3-methylcrotonyl-CoA carboxylase (MCC), and propionyl-CoA carboxylase (PCC), all of which participate in metabolic processes in mammals. However, biotin alone as a dietary supplement for healthy people without biotin deficiency or any inborn error of metabolism is unlikely to aid hair or nail health. Patel, D. P. et al., Skin Appendage Disord 3(3):166 (2017). For example, studies utilizing biotin supplementation alone for hair loss secondary to a laparoscopic sleeve gastrectomy (over a one-year period) provided mixed results with the conclusion indicating biotin on its own has low efficacy to prevent hair loss. Şen O. et al., J Laproendosc Adv Surg Tech A 31(3):296 (2021). In addition to hair and nail health, there is insufficient evidence that biotin alone aids skin health. Thompson, K. G. et al., J Am Acad Dermatol 84(4):1042 (2021).

According to embodiments described herein, when a composition comprising an active ingredient mixture comprising silicon and biotin is administered to a subject, the silicon and biotin act synergistically to improve the appearance of the subject's skin, hair, and nails.

In certain aspects, provided herein is a composition comprising an active ingredient mixture comprising silicon and biotin. In some embodiments, the mass ratio of the silicon to the biotin is from 1:50 to 50:1, from 1:20 to 20:1, from 1:10 to 10:1, from 1:5 to 5:1, from 1:3 to 3:1, from 1:2 to 2:1, from 2:3 to 3:2, or about 1:1. In certain embodiments, the mass ratio of the silicon to the biotin is from 2:1 to 5:1, from 5:2 to 7:2, or about 3:1. In some embodiments, the mass ratio of the silicon to the biotin is from 20:1 to 5:1, from 15:1 to 5:1, from 10:1 to 5:1, or from 7:1 to 6:1.

In certain embodiments, the mass ratio of the silicon to the biotin is from 1,000,000:1 to 1:2, from 100,000:1 to 1:2, from 10,000:1 to 1:2, from 1,000:1 to 1:2, from 100:1 to 1:2, from 10:1 to 1:2, from 1:1 to 1:2, from 1,000,000:1 to 1:1, from 100,000:1 to 1:1, from 10,000:1 to 1:1, from 1,000:1 to 1:1, from 100:1 to 1:1, from 10:1 to 1:1, from 1,000,000:1 to 10:1, from 100,000:1 to 10:1, from 10,000:1 to 10:1, from 1,000:1 to 10:1, from 100:1 to 10:1, from 1,000,000:1 to 100:1, from 100,000:1 to 100:1, from 10,000:1 to 100:1, from 1,000:1 to 100:1, from 1,000,000:1 to 1,000:1, from 100,000:1 to 1,000:1, from 10,000:1 to 1,000:1, from 1,000,000:1 to 10,000:1, from 100,000:1 to 10,000:1, or from 1,000,000:1 to 100,000:1.

In certain embodiments, the active ingredient mixture comprises from 0.01 mg to 1 g silicon, from 0.1 mg to 1 g silicon, from 1 mg to 1 g silicon, from 10 mg to 1 g silicon, from 100 mg to 1 g silicon, from 0.01 mg to 100 mg silicon, from 0.1 mg to 100 mg silicon, from 1 mg to 100 mg silicon, from 10 mg to 100 mg silicon, from 0.01 mg to 10 mg silicon, from 0.1 mg to 10 mg silicon, from 1 mg to 10 mg silicon, from 0.01 mg to 1 mg silicon, from 0.1 mg to 1 mg silicon, or from 0.01 mg to 0.1 mg silicon.

In some embodiments, the active ingredient mixture comprises from 0.5 mg to 500 mg silicon, from 1 mg to 300 mg silicon, from 1 mg to 50 mg silicon, from 2 mg to 40 mg silicon, from 3 mg to 30 mg silicon, from 4 mg to 20 mg silicon, from 5 mg to 20 mg silicon, or from 5 mg to 15 mg silicon.

In certain embodiments, the active ingredient mixture comprises from 0.001 mg to 100 mg biotin, from 0.01 mg to 100 mg biotin, from 0.1 mg to 100 mg biotin, from 1 mg to 100 mg biotin, from 10 mg to 100 mg biotin, from 0.001 mg to 10 mg biotin, from 0.01 mg to 10 mg biotin, from 0.1 mg to 10 mg biotin, from 1 mg to 10 mg biotin, from 0.001 mg to 1 mg biotin, from 0.01 mg to 1 mg biotin, from 0.1 mg to 1 mg biotin, from 0.001 mg to 0.1 mg biotin, from 0.01 mg to 0.1 mg biotin, or from 0.001 mg to 0.01 mg biotin.

In some embodiments, the active ingredient mixture comprises biotin in an amount less than 3 mg, less than 2.5 mg, less than 1 mg, less than 0.9 mg, less than 0.5 mg, less than 0.1 mg, less than 0.05 mg, or less than 0.045 mg.

In some embodiments, the active ingredient mixture comprises from 0.1 mg to 1 g biotin, from 0.5 mg to 500 mg biotin, from 1 mg to 300 mg biotin, from 1 mg to 50 mg biotin, from 1 mg to 40 mg biotin, from 1 mg to 30 mg biotin, from 1 mg to 20 mg biotin, or from 1 mg to 15 mg biotin.

In certain embodiments, the active ingredient mixture comprises from 5 mg to 20 mg silicon and from 0.01 mg to 1 mg biotin. In some embodiments, the active ingredient mixture comprises from 5 mg to 20 mg silicon and from 0.1 mg to 10 mg biotin. According to one or more embodiments, the active ingredient mixture comprises from 5 mg to 20 mg silicon and from 1 mg to 15 mg biotin. In some embodiments, the active ingredient mixture comprises from 8 mg to 12 mg silicon and from 8 mg to 12 mg biotin. In certain embodiments, the active ingredient mixture comprises about 10 mg silicon and about 10 mg biotin. According to one or more embodiments, the active ingredient mixture comprises from 5 mg to 20 mg silicon and from 1 mg to 5 mg biotin. In some embodiments, the active ingredient mixture comprises about 10 mg silicon and about 3 mg biotin. In certain embodiments, the active ingredient mixture comprises about 10 mg silicon and about 1.5 mg biotin. In further aspects, provided herein is a composition formulated to deliver silicon and biotin to a subject.

In some embodiments, the composition is formulated to deliver to a subject from 0.01 mg to 1 g silicon, from 0.1 mg to 1 g silicon, from 1 mg to 1 g silicon, from 10 mg to 1 g silicon, from 100 mg to 1 g silicon, from 0.01 mg to 100 mg silicon, from 0.1 mg to 100 mg silicon, from 1 mg to 100 mg silicon, from 10 mg to 100 mg silicon, from 0.01 mg to 10 mg silicon, from 0.1 mg to 10 mg silicon, from 1 mg to 10 mg silicon, from 0.01 mg to 1 mg silicon, from 0.1 mg to 1 mg silicon, or from 0.01 mg to 0.1 mg silicon.

In certain embodiments, the composition is formulated to deliver to a subject from 0.001 mg to 100 mg biotin, from 0.01 mg to 100 mg biotin, from 0.1 mg to 100 mg biotin, from 1 mg to 100 mg biotin, from 10 mg to 100 mg biotin, from 0.001 mg to 10 mg biotin, from 0.01 mg to 10 mg biotin, from 0.1 mg to 10 mg biotin, from 1 mg to 10 mg biotin, from 0.001 mg to 1 mg biotin, from 0.01 mg to 1 mg biotin, from 0.1 mg to 1 mg biotin, from 0.001 mg to 0.1 mg biotin, from 0.01 mg to 0.1 mg biotin, or from 0.001 mg to 0.01 mg biotin.

In some embodiments, the composition is formulated to deliver biotin in an amount less than 3 mg, less than 2.5 mg, less than 1 mg, less than 0.9 mg, less than 0.5 mg, less than 0.1 mg, less than 0.05 mg, or less than 0.045 mg.

In some embodiments, the composition is formulated to deliver from 5 mg to 20 mg silicon and from 0.01 mg to 1 mg biotin. In certain embodiments, the composition is formulated to deliver from 5 mg to 20 mg silicon and from 0.1 mg to 10 mg biotin. In some embodiments, the composition is formulated to deliver from 5 mg to 20 mg silicon and from 1 mg to 15 mg biotin to a subject. In some embodiments, the composition is formulated to deliver from 8 mg to 12 mg silicon and from 8 mg to 12 mg biotin to a subject. In certain embodiments, the composition is formulated to deliver about 10 mg silicon and about 10 mg biotin to a subject. In some embodiments, the composition is formulated to deliver from 5 mg to 20 mg silicon and from 5 mg to 15 mg biotin to a subject. According to one or more embodiments, the composition is formulated to deliver from 8 mg to 12 mg silicon and from 1 mg to 5 mg biotin to a subject. In some embodiments, the composition is formulated to deliver about 10 mg silicon and about 3 mg biotin to a subject. In certain embodiments, the composition is formulated to deliver about 10 mg silicon and about 1.5 mg biotin to a subject.

In some embodiments, the active ingredient mixture comprises magnesium. The magnesium may act synergistically with the silicon and the biotin in the active ingredient mixture. Magnesium plays a role in nucleic acid synthesis. Further, magnesium is a cofactor in various enzyme systems to regulate protein synthesis, muscle and nerve conduction, neuromuscular conduction, blood glucose, and blood pressure. Additionally, magnesium is an ion transporter for Ca and potassium (K) that works as a natural Ca antagonist. It has a regulatory role in the Mg—Ca channel gates and has a relaxation effect on endothelial cells and vascular smooth muscles. Magnesium also plays a role in nucleic acid synthesis, which is of significance in regions like the hair root as a site of frequent mitosis. Therefore, magnesium may support hair growth through increased blood flow to the hair and enhances the positive impact of biotin and silicon on hair growth. Additionally, many people consume insufficient magnesium in their diets. Accordingly, consumption of magnesium may ameliorate this deficiency. In certain embodiments, the active ingredient mixture comprises from 0.001 mg to 10 mg magnesium, from 0.01 mg to 10 mg magnesium, from 0.1 mg to 10 mg magnesium, from 1 mg to 10 mg magnesium, from 0.001 mg to 1 mg magnesium, from 0.01 mg to 1 mg magnesium, from 0.1 mg to 1 mg magnesium, from 0.001 mg to 0.1 mg magnesium, from 0.01 mg to 0.1 mg magnesium, or from 0.001 mg to 0.01 mg magnesium.

In some embodiments, the biotin is enriched with respect to D-biotin. According to one or more embodiments, the biotin is D-biotin. In certain embodiments, the biotin is a biotin salt. According to one or more embodiments, the biotin salt comprises ammonia or another organic base. In certain embodiments, the biotin salt comprises a metal. In some embodiments, the biotin salt comprises an alkali metal or an alkaline earth metal. According to one or more embodiments, the biotin salt comprises sodium, potassium, calcium, or magnesium. In certain embodiments, the biotin salt comprises magnesium.

In certain embodiments, the biotin salt has a water solubility of from 0.5 g/L to 500 g/L, from 1 g/L to 200 g/L, from 1 g/L to 100 g/L, from 1 g/L to 50 g/L, from 1 g/L to 20 g/L, from 2 g/L to 20 g/L, from 2 g/L to 15 g/L, from 5 g/L to 12 g/L, or from 6 g/L to 10 g/L.

In some embodiments, the biotin is magnesium biotinate. Magnesium biotinate has a water solubility about 40 times greater than biotin. Further, preclinical models have shown that magnesium biotinate is a bioavailable form of biotin that has superior absorption and greater uptake in tissue compared to D-biotin. In certain embodiments, the magnesium biotinate is prepared using the processes described in U.S. Patent Application Publication No. 2018/0071264, the entirety of which is incorporated herein by reference. In some embodiments, the molar ratio of magnesium to biotin is from 1:3 to 3:1. In certain embodiments, the molar ratio of magnesium to biotin is about 1:2. In some embodiments, the molar ratio of magnesium to biotin is about 1:1. According to one or more embodiments, the magnesium biotinate is magnesium D-biotinate.

In some embodiments, the active ingredient mixture comprises arginine. In certain embodiments, arginine may act synergistically with the silicon and the biotin in the active ingredient mixture. Dietary supplementation with arginine has been shown to facilitate wound healing, enhance insulin sensitivity, collagen deposition, cell proliferation, T-lymphocyte function, protein synthesis, and promote positive nitrogen balance. Arginine is known to play a significant role in several metabolic processes. Arginine by itself is known to be a vasodilator and positive promoter of nitric oxide production. L-arginine is the substrate for the enzyme Nitric Oxide Synthase (NOS), which is responsible for the endothelial production of nitric oxide. Dietary supplementation with L-arginine has been shown to have health benefits, including significantly improving endothelial function in individuals even without vascular disease (healthy subjects). Blood flow to the skin is neurally controlled by the adrenergic vasoconstrictor system and an active vasodilator system. The vasodilator pathways mediate downstream nitric oxide dependent vasodilatation, which is needed for the full reflex vasodilatory response in humans, the nutritional driver of NO production is the amino acid arginine. Understanding that L-arginine is the common substrate for both Nitric Oxide Synthase (NOS) and arginase is important, as arginase also catalyzes the conversion of arginine to urea and ornithine, which is the precursor to proline. Proline has importance in skin and other tissue integrity, especially for collagen synthesis. Arginine, in addition to being able to be converted into proline, also can be incorporated into collagen itself. The arginine-derived nitric oxide and arginine itself may have an important role in the overall health and appearance of skin.

In certain embodiments, the active ingredient mixture comprises an arginine silicate complex. According to one or more embodiments, the active ingredient mixture comprises a polyol. In some embodiments, the active ingredient mixture comprises inositol.

In certain embodiments, the active ingredient mixture comprises an inositol-stabilized arginine-silicate complex (ASI). ASI has beneficial effects on vascular and bone health. Further, the use of arginine in complexes with inositol and silicon increases arginine and silicon absorption. Additionally, ASI has been shown to enhance blood flow, which improves nutrient delivery to skin, hair, and nails. In some embodiments, ASI enhances the supply of nutrients to the hair by arginine through vasodilatation of the hair follicle via nitric oxide and silica contained in ASI supporting hair growth through the ODC enzyme. In certain embodiments, ASI prolongs the anagen phase through growth factors and thereby increases hair density and anagen hair percentages.

In some embodiments, the active ingredient mixture comprises ASI and inositol separate from (i.e., not associated with) the arginine silicate complex.

In some embodiments, the ASI is prepared using the processes described in U.S. Pat. Nos. 5,707,970, 6,803,456, and 11,103,000, each of which are incorporated by reference herein in their entirety.

In certain embodiments, the ASI has a molar ratio of arginine to silicate of from about 0.5:1 to about 2:1, from about 0.75:1 to about 1.25:1, from about 0.8:1 to about 1.2:1, or about 1:1. In some embodiments, the ASI has a molar ratio of arginine to inositol of from about 1:1 to about 4:1, from about 1.25:1 to about 3:1, from about 1.5:1 to about 3:1, or about 2:1. According to one or more embodiments, the ASI has a molar ratio of arginine to silicate to inositol of about 3:3:1 or about 2:2:1. In certain embodiments, the ASI has a molar ratio of arginine to silicate to inositol of about 2:2:1.

In certain embodiments, the ASI comprises from 48% and 51% arginine, from 7% to 9% silicon, and from 23% to 27% inositol. In some embodiments, the ASI comprises about 50% arginine, about 8% silicon, and about 25% inositol.

In further aspects, provided herein is a composition comprising an active ingredient mixture comprising ASI and magnesium biotinate.

According to one or more embodiments, the mass ratio of the ASI to the magnesium biotinate is from 10,000,000:1 to 1:1, from 1,000,000:1 to 1:1, from 100,000:1 to 1:1, from 10,000:1 to 1:1, from 1,000:1 to 1:1, from 100:1 to 1:1, from 10:1 to 1:1, from 10,000,000:1 to 10:1, from 1,000,000:1 to 10:1, from 100,000:1 to 10:1, from 10,000:1 to 10:1, from 1,000:1 to 10:1, from 100:1 to 10:1, from 10,000,000:1 to 100:1, from 1,000,000:1 to 100:1, from 100,000:1 to 100:1, from 10,000:1 to 100:1, from 1,000:1 to 100:1, from 10,000,000:1 to 1,000:1, from 1,000,000:1 to 1,000:1, from 100,000:1 to 1,000:1, from 10,000:1 to 1,000:1 from 10,000,000:1 to 10,000:1, from 1,000,000:1 to 10,000:1, from 100,000:1 to 10,000:1, from 10,000,000:1 to 100,000:1, from 1,000,000:1 to 100,000:1, or from 10,000,000:1 to 1,000,000:1.

In certain embodiments, the mass ratio of the ASI to the magnesium biotinate is from 2:1 to 100:1, from 3:1 to 100:1, from 5:1 to 100:1, from 5:1 to 50:1, from 5:1 to 30:1, from 7:1 to 20:1, from 9:1 to 18:1, from 10:1 to 15:1, or from 12:1 to 13:1. In some embodiments, the mass ratio of the ASI to the magnesium biotinate is from 20:1 to 70:1, from 30:1 to 50:1, from 35:1 to 50:1, from 37:1 to 47:1, from 40:1 to 45:1, or about 42:1. In some embodiments, the active ingredient mixture ASI in an amount of from 1 mg to 1 g, from 10 mg to 1 g, from 100 mg to 1 g. from 1 mg to 100 mg, from 10 mg to 100 mg, or from 1 mg to 10 mg. In certain embodiments, the active ingredient mixture comprises a daily dosage of ASI in an amount of from 10 mg to 500 mg, from 50 mg to 300 mg, from 100 mg to 200 mg, or about 150 mg.

In certain embodiments, the active ingredient mixture comprises magnesium biotinate in an amount of from 0.001 mg to 100 mg, from 0.01 mg to 100 mg, from 0.1 mg to 100 mg, from 1 mg to 100 mg, from 10 mg to 100 mg, from 0.001 mg to 10 mg, from 0.01 mg to 10 mg, from 0.1 mg to 10 mg, from 1 mg to 10 mg, from 0.001 mg to 1 mg, from 0.01 mg to 1 mg, from 0.1 mg to 1 mg, from 0.001 mg to 0.1 mg, from 0.01 mg to 0.1 mg, or from 0.001 mg to 0.01 mg.

In certain embodiments, the active ingredient mixture comprises from 50 mg to 300 mg ASI and from 1 mg to 15 mg magnesium biotinate. In some embodiments, the active ingredient mixture comprises from 50 mg to 300 mg ASI and from 0.01 mg to 1 mg magnesium biotinate.

In some embodiments, the composition is formulated as a tablet, a gummy, a gel, a chewable tablet, a dissolvable tablet, a dissolvable sheet, a microencapsulated capsule, an elixir, a syrup, a sachet, a liquid, a mouthwash, a tincture or a paste. In certain embodiments, the composition is formulated for sublingual absorption. In certain embodiments, the composition is formulated for oral administration. Oral administration can allow for the absorption of the bioactive components of the composition via the mucous membranes of the mouth or via the intestinal tract. In some embodiments, the composition is administered orally as a gel. In some embodiments, the composition is administered as a dissolvable tablet that dissolves in the mouth. In some embodiments, the composition is administered as a gel-like sheet that dissolves on the tongue. In some embodiments, the composition is administered as a liquid, such as a mouthwash, ready-to-drink beverage, or a powder that can be dissolved in a liquid, such as water. In some embodiments, the composition is administered as a tincture. In certain embodiments, the composition is formulated as a dispersible powder, a beverage, a hard capsule, or a soft capsule. According to one or more embodiments, the composition is formulated for extended release, controlled release, or a combination thereof. In some embodiments, the composition is formulated for sustained or prolonged release. In certain embodiments, the composition comprises a pharmaceutically acceptable excipient.

In certain embodiments, the composition is formulated for topical administration. In some embodiments, the composition is formulated as a hair gel, a shampoo, a conditioner, a cream, a lotion, or a salve. According to one or more embodiments, the composition is formulated as an adhesive sheet. In some embodiments, the composition is formulated as a cream. In some embodiments, the composition is formulated as a hair serum. According to one or more embodiments, the composition is formulated as a hair gel. In certain embodiments, the composition is formulated as a lotion. According to one or more embodiments, the composition is formulated as a sunscreen. In some embodiments, the composition is formulated as a sunscreen spray. In certain embodiments, the composition is formulated as a sunscreen lotion.

In some embodiments, the composition comprises the active ingredient mixture in a concentration of from 0.01 wt. % to 100 wt. %, from 0.1 wt. % to 100 wt. %, from 0.1 wt. % to 80 wt. %, from 0.1 wt. % to 50 wt. %, from 0.5 wt. % to 20 wt. %, from 1 wt. % to 20 wt. %, from 1 wt. % to 10 wt. %, from 5 wt. % to 15 wt. %, from 10 wt. % to 70 wt. %, from 0.5 wt. % to 5 wt. %, or from 0.1 wt. % to 2 wt. %.

Compositions disclosed herein, pharmaceutically acceptable salts of the compositions disclosed herein, and pharmaceutically acceptable solvates of compositions disclosed herein can be co-formulated alone but, in human therapy, will generally be administered in admixture with a suitable pharmaceutical excipient diluent or carrier selected with regard to the intended route of administration and standard pharmaceutical practice. For example, compositions can be administered orally, buccally or sublingually in the form of tablets, capsules (including soft gel capsules), ovules, elixirs, solutions or suspensions, which may contain flavoring or coloring agents, for immediate-, delayed-, modified-, sustained-, or controlled-release delivery applications. Modified release dosage forms can contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release release dosage forms may contain one or a combination of release rate modifying excipients. Release rate modifying excipients can be present both within the dosage form i.e. within the matrix, and/or on the dosage form i.e. upon the surface or coating.

A tablet, capsule, gel cap, or caplet can contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethyl cellulose (HPMC), hydroxypropylcellulose (HPC), sucrose, gelatin and acacia. Additionally, lubricating agents such as magnesium stearate, stearic acid, glyceryl behenate and talc can also be included.

In further aspects, provided herein is a method of improving the appearance of a subject's skin, hair, or nails comprising administering a composition comprising an active ingredient mixture comprising silicon and biotin to the subject.

In some embodiments, the method improves the appearance of a subject's skin. In certain embodiments, the method improves the appearance of a subject's hair. According to one or more embodiments, the method improves the appearance of a subject's nails. In certain embodiments, the method improves the appearance of a subject's skin and nails, skin and hair, or hair and nails. According to one or more embodiments, the method improves the appearance of a subject's skin, hair, and nails.

In some embodiments, the method improves skin elasticity, skin hydration, skin texture (e.g., reduces skin roughness or skin scaling), facial wrinkle depth, skin inflammation, hair pigmentation, hair thickness, hair density, hair volume, hair shine, hair strength, nail strength, nail growth, hair growth, or a combination thereof.

In certain embodiments, the method increases hair density. In some embodiments, the method increases the duration of the anagen stage of hair growth. According to one or more embodiments, the method increases anagen ratio. In certain embodiments, the method decreases telogen ratio.

In further aspects, provided herein is a method of treating or preventing a condition in a subject comprising administering a composition comprising an active ingredient mixture comprising silicon and biotin to the subject.

In some embodiments, the condition comprises deterioration of the subject's hair, skin, or nails. In certain embodiments, the condition arises from a disease. In some embodiments, the condition comprises a symptom of a disease. In certain embodiments, the condition comprises a side effect from a treatment for a disease.

In certain embodiments, the condition is a genetic condition, menopause, chronic stress, aging, or chronic inflammation. In some embodiments, the condition comprises a side effect of a medication. In certain embodiments, the condition comprises a side effect of a medication administered to treat cancer, depression, or heart disease.

In some embodiments, the condition comprises hair thinning, hair loss, skin wrinkles, spontaneously aged skin, photodamaged skin, photoaged skin, irregular skin pigmentation, or nail brittleness.

In certain embodiments, the condition comprises spontaneously aged skin. Internal skin aging, termed ‘spontaneous aging,’ is the physiological changing of the skin affected by genetic factors and occurs naturally over time.

In certain embodiments, the condition comprises photoaged skin. Photoaging from chronic ultraviolet (UV) exposure leads to a complex skin-changing process that occurs predominantly on cutaneous surfaces exposed to the sun. Photoaging mechanisms include free radicals, which lead to the accumulation of reactive oxygen species (ROS). ROS mediates harmful post-translational properties on aging skin by direct chemical alterations to DNA, cell lipids, and dermal matrix proteins, including collagens. All these processes result in an irregular and non-functional accumulation of elastic fibers in the skin. Clinical manifestations of photoaging include wrinkles, telangiectasias, laxity, loss of translucency, and various pigmented spots such as freckles and solar lentigines. Similar to photoaging, chronic exposure to solar radiation causes multiple skin disorders, including sunburn, irregular pigmentation, and skin cancer, specifically non-melanoma skin cancers.

In certain embodiments, the condition comprises photodamaged skin caused by exposure to UVB radiation. UV radiation includes UVA and UVB radiation, and without being bound by any particular theory, it is believed that UVB radiation is primarily responsible for degrading skin health, including damage from sunrays such as photoaging, wrinkles, sunburns, and skin cancers. Without intending to be bound by any particular theory, an active ingredient mixture comprising ASI and magnesium biotinate may treat or prevent photodamaged skin caused by UVB radiation by regulating MMPs, mTOR, and inflammatory markers, including TNF-α, NFκB, IL-6, IL-8, and COX-2 and apoptotic pathways. In some embodiments, the methods described herein alleviate macroscopic skin damage from UV exposure. In certain embodiments, the methods described herein alleviate histopathological skin damage from UV exposure.

In certain embodiments, the composition is administered orally to the subject. In some embodiments, the composition is administered topically to the subject.

In some embodiments, the composition is conjunctively administered with a second composition. In some embodiments, the second composition is formulated to be administered through a different route than the composition. In certain embodiments, the second composition comprises silicon, biotin, or a combination thereof. According to one or more embodiments, the second composition comprises magnesium biotinate or ASI. In some embodiments, the second composition comprises magnesium biotinate and ASI. In certain embodiments, the second composition is a composition according to any of the embodiments described herein. According to one or more embodiments, the second composition comprises ASI and does not comprise magnesium biotinate. In some embodiments, the second composition comprises magnesium biotinate and does not comprise ASI. In certain embodiments, the second composition is formulated for oral administration. In some embodiments, the second composition is formulated as a dispersible powder, a beverage, a gummy, a gel, a tablet, a hard capsule, or a soft capsule. In some embodiments, the second composition is formulated for topical administration. In certain embodiments, the second composition is formulated according to embodiments described in U.S. Patent Application Publication No. 2021/0338606, the entirety of which is incorporated herein by reference. In certain embodiments, the second composition is formulated as a hair serum, a cream, or a lotion. According to one or more embodiments, the second composition is formulated as a sunscreen. In some embodiments, the second composition is a hair serum comprising ASI and magnesium biotinate. In certain embodiments, the second composition is a cream comprising ASI and magnesium biotinate. In certain embodiments, the second composition is a cream comprising magnesium biotinate. In some embodiments, the second composition is a cream comprising magnesium biotinate in a concentration of from 0.1 wt. % to 50 wt. %, from 0.5 wt. % to 20 wt. %, from 1 wt. % to 10 wt. %, from 1 wt. % to 5 wt. %, from 1 wt. % to 3 wt. %, or about 2 wt. %. In certain embodiments, the second composition is a lotion comprising magnesium biotinate in a concentration of from 0.1 wt. % to 50 wt. %, from 0.5 wt. % to 20 wt. %, from 1 wt. % to 10 wt. %, from 1 wt. % to 5 wt. %, from 1 wt. % to 3 wt. %, or about 2 wt. %.

In some embodiments, the composition is administered orally and the second composition is conjunctively administered topically to the subject. In certain embodiments, the composition is administered topically and the second composition is conjunctively administered orally to the subject.

In certain embodiments, the composition is administered to the subject at least once per day. In some embodiments, a daily dosage of the active ingredient mixture comprises from 0.01 mg to 1 g silicon, from 0.1 mg to 1 g silicon, from 1 mg to 1 g silicon, from 10 mg to 1 g silicon, from 100 mg to 1 g silicon, from 0.01 mg to 100 mg silicon, from 0.1 mg to 100 mg silicon, from 1 mg to 100 mg silicon, from 10 mg to 100 mg silicon, from 0.01 mg to 10 mg silicon, from 0.1 mg to 10 mg silicon, from 1 mg to 10 mg silicon, from 0.01 mg to 1 mg silicon, from 0.1 mg to 1 mg silicon, or from 0.01 mg to 0.1 mg silicon.

In certain embodiments, the active ingredient mixture comprises from 0.5 mg to 500 mg silicon, from 1 mg to 300 mg silicon, from 1 mg to 50 mg silicon, from 2 mg to 40 mg silicon, from 3 mg to 30 mg silicon, from 4 mg to 20 mg silicon, from 5 mg to 20 mg silicon, or from 5 mg to 15 mg silicon.

In certain embodiments, a daily dosage of the active ingredient mixture comprises from 0.001 mg to 100 mg biotin, from 0.01 mg to 100 mg biotin, from 0.1 mg to 100 mg biotin, from 1 mg to 100 mg biotin, from 10 mg to 100 mg biotin, from 0.001 mg to 10 mg biotin, from 0.01 mg to 10 mg biotin, from 0.1 mg to 10 mg biotin, from 1 mg to 10 mg biotin, from 0.001 mg to 1 mg biotin, from 0.01 mg to 1 mg biotin, from 0.1 mg to 1 mg biotin, from 0.001 mg to 0.1 mg biotin, from 0.01 mg to 0.1 mg biotin, or from 0.001 mg to 0.01 mg biotin.

In some embodiments, a daily dosage of the biotin is less than 3 mg, less than 2.5 mg, less than 1 mg, less than 0.9 mg, less than 0.5 mg, less than 0.1 mg, less than 0.05 mg, or less than 0.045 mg.

In some embodiments, a daily dosage of the active ingredient mixture comprises from 0.1 mg to 1 g biotin, from 0.5 mg to 500 mg biotin, from 1 mg to 300 mg biotin, from 1 mg to 50 mg biotin, from 1 mg to 40 mg biotin, from 1 mg to 30 mg biotin, from 1 mg to 20 mg biotin, or from 1 mg to 15 mg biotin. According to one or more embodiments, a daily dosage of the active ingredient mixture comprises from 5 mg to 20 mg silicon and from 5 mg to 15 mg biotin.

In certain embodiments, a daily dosage of the active ingredient mixture comprises from 5 mg to 20 mg silicon and from 0.01 mg to 1 mg biotin. In some embodiments, a daily dosage of the active ingredient mixture comprises from 5 mg to 20 mg silicon and from 0.1 mg to 10 mg biotin. In certain embodiments, a daily dosage of the active ingredient mixture comprises from 5 mg to 20 mg silicon and from 1 mg to 15 mg biotin. In some embodiments, a daily dosage of the active ingredient mixture comprises from 8 mg to 12 mg silicon and from 8 mg to 12 mg biotin. In certain embodiments, a daily dosage of the active ingredient mixture comprises about 10 mg silicon and about 10 mg biotin. According to one or more embodiments, a daily dosage of the active ingredient mixture comprises from 8 mg to 12 mg silicon and from 1 mg to 5 mg biotin. In some embodiments, a daily dosage of the active ingredient mixture comprises about 10 mg silicon and about 3 mg biotin. In certain embodiments, a daily dosage of the active ingredient mixture comprises about 10 mg silicon and about 1.5 mg biotin.

In certain embodiments, the methods described herein increase one or more of Mg, Fe, Zn, Cu, Si, biotin, and arginine concentrations in the subject's serum. In some embodiments, the methods described herein increase skin hydroxyproline levels. In certain embodiments, the methods described herein increase skin biotinidase levels. In some embodiments, the methods described herein decrease skin elastase activity. In certain embodiments, the methods described herein decrease skin malondialdehyde (MDA) concentration. In some embodiments, the methods described herein increase skin levels of biotin-dependent carboxylases. In certain embodiments, the methods described herein decrease mammalian target of rapamycin pathways. In some embodiments, the methods described herein decrease matrix metalloproteinase protein levels. In certain embodiments, the methods described herein decrease levels of inflammatory factors. In some embodiments, the methods described herein decrease levels of Bax and caspase-3. In some embodiments, the methods described herein increase levels of anti-apoptotic protein Bel-2. In some embodiments, the methods described herein increase protein levels of VEGF and SIRT1. In certain embodiments, the methods described herein decrease levels of mTORC2 (ser2481), p-p70S6K, p4E-BP1, MMP-1, MMP-3, or a combination thereof. In some embodiments, the methods described herein decrease inflammatory factors, such as TNF-α, NFκB, IL-6, IL-8, COX-2, or a combination thereof. In some embodiments, the methods described herein downregulate p-JNK and p-p38 levels. In certain embodiments, the methods described herein block the activation of the AP-1. In some embodiments, the methods described herein increase skin carboxylases such as ACC1, ACC2, PC, PCC, MCC, or a combination thereof.

In certain embodiments, the methods described herein prevent the overproduction of TNF-α, NF-κB, IL-6, IL-8, COX-2, or a combination thereof. In some embodiments, the methods described herein suppress UVB-induced dermal inflammatory infiltrate. In certain embodiments, the methods described herein prevent a decrease in Bcl-2. In some embodiments, the methods described herein regulate MMPs, mTOR, pathways, and inflammatory markers, including TNF-α, NF-κB, IL-6, IL-8, COX-2 (that may be associated with suppression of JNK, p38 MAPK, and AP-1), apoptosis, or a combination thereof. In some embodiments, the methods described herein suppress p-JNK, p-38, c-Jun, c-Fos, or a combination thereof in skin tissues. In certain embodiments, the methods described herein regulate the AP-1 and MAPK pathways. In certain embodiments, the methods described herein decrease levels of Bax and caspase-3 while increasing levels of anti-apoptotic protein Bcl-2.

In some embodiments, the methods described herein induce Wnt signaling. In some embodiments, the methods described herein induce β-catenin signaling. The Wnt signal pathway and β-catenin are important signals that form the hair follicle and enable hair follicle regeneration. The activation of β-catenin in the dermal papilla regulates signal pathways involving FGF and IGF. In certain embodiments, the methods described herein increase the levels of KGF, HGF, VEGF, or a combination thereof. In some embodiments, the methods described herein increase the levels of IGF-1, FGF, or a combination thereof. FGF has been shown to stimulate the anagen phase and thereby hair growth. KGF (a member of the FGF family) and HGF are other signals known to prolong the anagen phase. It has been reported that VEGF stimulates vasculogenesis and angiogenesis and supplies nutrients to the hair follicle by increasing the follicle diameter.

In certain embodiments, the methods described herein induce SIRT-1. Sirtuins are nicotinamide adenine dinucleotide (NAD)-dependent deacylase enzymes known for their antiaging characteristics. They play a role in regulating the cellular response to stress, DNA repair and metabolism, and tumorigenesis. SIRT-1 positively affects glucose-induced insulin response. SIRT-1 has been shown to enable mitochondrial homeostasis against the stress caused by TNF-α-dependent mediators on the hair follicle and increased the stem cells in the hair follicle as a way of protection. The decrease in hair pigmentation in chronological aging might be prevented by increasing the levels of SIRT-1. Arginine has been shown to increase the expression of SIRT-1 and MMP2, reduce myocardial fibrosis and prevent cell apoptosis in streptozotocin-induced diabetic rats.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art.

Chemistry terms used herein, unless otherwise defined herein, are used according to conventional usage in the art, as exemplified by “The McGraw-Hill Dictionary of Chemical Terms,” Parker S., Ed., McGraw-Hill, San Francisco, C.A. (1985).

The term “subject” refers to animals which can be treated using the compositions and methods of the present disclosure. Examples of animals include mammals, such as mice, rabbits, rats, horses, goats, dogs, cats, pigs, cattle, sheep, and primates (e.g. chimpanzees, gorillas, and, preferably, humans). In some embodiments, the term “subject” as used herein refers to an animal. In some embodiments, the subject is a mammal. In certain embodiments, the subject is a mouse, a rabbit, a rat, a horse, a goat, a dog, a cat, a pig, a cattle, a sheep, or a primate. According to one or more embodiments, the subject is a chimpanzee, a gorilla, or a human. In some embodiments, the subject is a human. In certain embodiments, the human is female. According to one or more embodiments, the human is male. In some embodiments, a female subject (such as a female human) responds differently to administration of the compositions described herein than a male subject (such as a male human) and therefore requires a different dose (such as a different daily dose).

As used herein, the term “hair” refers to keratinous filaments protruding from the skin of the subject. In some embodiments, the hair is head hair. In certain embodiments, the hair is androgenic hair. In some embodiments, the hair is facial hair.

In some embodiments, the compositions disclosed herein are in the form of pharmaceutically effective salts. The phrase “pharmaceutically acceptable salt(s),” is art recognized and, as used herein includes, but is not limited to, salts of acidic or basic groups that may be present in the compositions disclosed herein. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds present in the compositions disclosed hererein that include an amino moiety also can form pharmaceutically acceptable salts with various amino acids, in addition to the acids mentioned above. Compounds present in the compositions disclosed herein that are acidic in nature are capable of forming base salts with various pharmacologically acceptable cations. Non limiting examples of such salts include alkali metal or alkaline earth metal salts and, particularly, calcium, magnesium, sodium lithium, zinc, potassium, silicon, phosphorus and iron salts. In certain embodiments, “pharmaceutically acceptable salt” or “salt” is used herein to refer to an acid addition salt or a basic addition salt which is suitable for or compatible with the treatment of patients.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients; such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. In one embodiment, the pharmaceutically acceptable carrier is suitable for intravenous administration. In another embodiment, the pharmaceutically acceptable carrier is suitable for locoregional injection. In some embodiments, the phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “biotin” as used herein refers to a compound having the following structure:

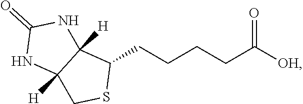 [SMILES: O=C(O)CCCC[C@@H]1SC[C@@H]2NC(=O)N[C@@H]21]

a salt thereof, a stereoisomer thereof, or a derivative thereof that converts into the compound, the salt thereof, or the stereoisomer thereof in vivo.

The term “silicon” as used herein refers to the element Si in any oxidation state.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

“Treating” a condition, patient, or population of patients refers to taking steps to obtain beneficial or desired results, including clinical results. As used herein, “treatment” is an approach for obtaining beneficial or desired results, including clinical results. Beneficial or desired clinical results can include, but are not limited to, alleviation or amelioration of one or more symptoms or conditions, reduction in severity of condition, stabilized (i.e. not worsening) state of condition, preventing spread of condition, delay or slowing of condition progression, amelioration or palliation of the condition state, and remission (whether partial or total), whether detectable or undetectable. “Treatment” can also mean prolonging survival as compared to expected survival if not receiving treatment. Successful treatment may be evaluated, e.g., by evaluating the physical characteristics and/or pathology of the subjects or population of subjects to which a composition has been administered relative to a control subject or population of subjects.

As described herein, the term “effective” can mean an amount that is capable of achieving the results specified in the claims and the disclosure as described herein. The term “effective” can refer to a “pharmaceutically effective amount” or a “nutraceutically effective amount” as these terms be used interchangeably as set forth herein. One of skill in the art would immediately envisage the meaning and scope of these terms depending upon their usage in the surrounding context. For further context, a pharmaceutically effective amount can often refer to an amount that is administered to achieve a therapeutic result whereas a nutraceutically effective amount can be an amount of a composition that is administered to maintain healthy levels of a particular biomarker, physical characteristic, or other health indicator. In some embodiments, an effective amount can refer to an amount that is capable of maintaining homeostatic levels of a particular characteristic of a subject or returning a subject to homeostatic levels of a particular characteristic. In some embodiments, a “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

The term “preventing.” when used in relation to a condition, such as a local recurrence (e.g., pain), includes administration of a composition which reduces the frequency of, delays the onset of, reduces the severity of, or avoids symptoms of a condition in a subject or population of subjects relative to a subject or population of subjects which does not receive the composition. Successful prevention may be evaluated, e.g., by evaluating the physical characteristics and/or pathology of the subjects or population of subjects to which a composition has been administered relative to a control subject or population of subjects.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different compositions such that the second composition is administered while the previously administered composition is still effective in the body (e.g., the two compositions are simultaneously effective in the subject, which may include synergistic effects of the two compositions). For example, the different compositions can be administered either concomitantly or sequentially. Thus, a subject that receives such conjoint administration can benefit from a combined effect of the different compositions.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

### EXAMPLES

The invention now being generally described, it will be more readily understood by reference to the following examples, which are included merely for purposes of illustration of certain aspects and embodiments of the present invention, and are not intended to limit the invention.

### Example 1

The effects of administering a composition comprising an active ingredient mixture of inositol-stabilized arginine silicate complex (ASI) and magnesium biotinate (MgB) on the prevention of skin damage after UVB exposure in rats was studied. Forty-nine Sprague-Dawley rats were randomized into one of the following groups: (1) NC, normal control, (2) SC, shaved control, (3) UVB (exposed to UVB radiation), (4) ASI+MgB-L (Low Dose), (5) ASI+MgB-H (High Dose), (6) ASI+MgB-L+MgB cream, (7) ASI+MgB-H+MgB cream. The results showed that ASI+MgB treatment alleviated the macroscopic and histopathological damages in the skin of rats caused by UVB exposure. Skin elasticity evaluation showed a similar trend. ASI+MgB increased serum Mg, Fe, Zn, Cu. Si, biotin, and arginine concentrations and skin hydroxyproline and biotinidase levels while decreasing skin elastase activity (p<0.05) and malondialdehyde (MDA) concentration (p<0.001). Moreover, ASI+MgB treatment increased skin levels of biotin-dependent carboxylases (ACC1, ACC2, PC, PCC, MCC) and decreased mammalian target of rapamycin (mTOR) pathways and matrix metalloproteinase protein levels by the regulation of the activator protein 1 (AP-1), and mitogen activated protein kinases (MAPKs) signaling pathways. In addition, ASI+MgB caused lower levels of inflammatory factors, including TNF-α, NFκB, IL-6. IL-8, and COX-2 in the skin samples (p<0.05). The levels of Bax and caspase-3 were increased, while anti-apoptotic protein Bcl-2 was decreased by UVB exposure, which was reversed by ASI+MgB treatment. These results show that treatment with ASI and MgB protects against skin damage by improving skin appearance, elasticity, inflammation, apoptosis, and overall health.

### Animals and Experimental Procedures

A total of 49 male Sprague-Dawley rats (age: 8 weeks, mean weight: 180±20 g) purchased from Firat University Experimental Research Center (FUDAM) were used in the study. The rats were maintained in a controlled environment with a 12:12 light-dark cycle, room temperature of 22±2 C and humidity of 55%±10, and had ad libitum access to feed and water.

The dorsal hair of rats was clipped under anesthesia (2 cm in width×6 cm in length) prior to the experiment. Forty-nine rats were randomly divided into seven groups with seven rats each, as shown in Table 1:1) NC (normal control; no UVB radiation), 2) SC (shaved control; shaved and no UVB radiation), 3) UVB group (shaved and received UVB radiation), 4) ASI+MgB-L [shaved, received UVB radiation, ASI (140 mg human equivalent dose (HED)=2.2 mg ASI/rat/d) and low dose MgB (1.5 mg HED of biotin=24 μgMgB/rat/d], 5) ASI+MgB-H [shaved, received UVB radiation, ASI and high dose MgB (10 mg HED of biotin=160 μg MgB/rat/d)], 6) ASI+MgB-L+MgB-C [shaved received UVB radiation, ASI, low dose MgB and MgB cream application (2% MgB)], 7) ASI+MgB-H+MgB-C (shaved, received UVB radiation, ASI and high dose MgB and MgB cream application).

The diet used in the study was adapted to comprise spray-dried egg whites as the single protein source. Avidin protein in egg white binds approximately 1.44 mg biotin/kg purified diet, inhibiting biotin absorption. All treatments (ASI and MgB) were supplemented daily as an oral supplement, and UVB irradiation was performed five times per week for ten weeks. The normal control shaved control, and UVB rats were orally administered with saline. The oral and topical dosages of both agents were determined as proposed in the literature and as would have been understood by the skilled artisan.

The ASI and MgB were provided by JDS Therapeutics, LLC (Harrison, NY, United States). The ASI can be prepared using the processes described in U.S. Pat. Nos. 5,707,970, 6,803,456, and 11,103,000. The MgB can be prepared using the processes described in U.S. Patent Application Publication No. 2018/0071264. Both ASI and MgB were dissolved in drinking water and were administered via oral gavage. In the control group, saline was administered by oral gavage after being dissolved in drinking water. Oral gavage and 2% MgB cream were administered 2 h and 30 min prior to the experiment, respectively (Table 1).

### UVB Irradiation (Photoaging Modeling)

For the photoaged rat model, rats were exposed to UVB lamps with UVB with emission spectrums between 290 and 320 nm (peak at 310-315 nm) (Waldman UV800, Germany) and UVA emissions in the range of 320-400 nm (peak at 365) four times per week. To determine the minimal erythema dose (MED) of rats in each group, rats were exposed to UVB at doses of 20, 30, 40, 60, 80, and 100 mJ/cm² (approximate dosage increase=40%) on a total area of 24 cm² on the lower back, which comprised a total of six sites measuring 2 cm×2 cm. MED was accepted as the amount of UV radiation that produced a minimal erythematous reaction with well-demarcated borders 24 h after UVB exposure. Prior to the experiment's initiation, the average MED of the seven rats in each group was accepted as the baseline MED for each group. Throughout UVB exposure, the rats were kept in the incubator at a 20-30 cm distance from the UVB lamp, and UVB exposure was performed four times a week. The initial radiation dose was 1 MED in the first and second weeks and was increased by 0.2 MED/week over the remaining 10 weeks (range between weeks 3 and 1=1.2-2.6 MED). UV energy was detected with a UV radiometer. The same procedure was performed in the non-irradiated control group; however, the UVB was switched off. ASI and MgB were administered by oral gavage 2 h prior to the study, and topical 2% MgB cream was administered 30 min before the study.

After successful modeling, the rats were anesthetized; their dorsal skin was photographed. At the end of the experiment, all the rats were sacrificed by cervical dislocation, and blood and dorsal skin samples removed from the irradiated area were collected for further analyses. Blood samples were centrifuged at 3000×g for 10 min, and the serum was carefully removed for further analysis. The sera and tissue samples were stored in a deep freeze (Hettich, Germany) at 0° C. until analysis. The skin of the back of the animals was observed daily during the irradiation period.

### Evaluation of Dorsal Skin

A blinded observer assessed noticeable variation of rats' dorsal skin according to assessment criteria. Skin elasticity was measured by performing the pinch test according to the adapted protocol described by Tsukahara et al. The dorsal skin at the midline of the rat was picked up with fingers as much as possible until the lower limbs were almost off the ground, and then the rat was released from the fingers. Skin elasticity was defined as skin recovery time between the onset and disappearance of the pinch, whereby longer recovery time was accepted to indicate lower skin elasticity.

### Biochemical Analysis

Serum glucose, urea-N (BUN), and creatinine concentrations and activities of alanine aminotransferase (ALT) and aspartate aminotransferase (AST) were analyzed with an automated chemistry analyzer (Samsung LABGEO PT10V, Samsung Electronics Co., Suwon, Korea). For hydroxyproline analyses, the dorsal skin tissues were homogenized using 2 ml of 1 N acetic acid, and the homogenates were centrifuged (3,000 g, 10 min). The hydroxyproline levels were measured in the supernatants using a commercially available assay kit according to the manufacturer's instructions (Diagnostic Systems Laboratories, TX, United States). The intra- and inter-assay coefficients of variations (CV) were 4.3 and 6.5%. The tissue was also homogenized and solubilized in 0.1% Triton-X 100, 0.2 M Tris-HCl (pH 8.0) buffer, followed by ultrasonication and by centrifugation (2000 g×20 min) to obtain supernatants for the elastase enzyme assay. The activity was detected using a commercially available assay kit (Diagnostic Systems Laboratories, TX, United States) according to the manufacturer's instructions. The inter- and intra-assay constants were 3.6 and 6.4%. Skin activity of biotinidase was detected using the colorimetric method by measuring p-aminobenzoate liberation from N-biotinyl-p-aminobenzoate. Biotinidase activity is expressed as mU/100 mg protein.

To determine the concentrations of serum levels of Mg, silicon (Si), iron (Fe), copper (Cu), and zinc (Zn) in the sera and tissue samples, 0.3 g of skin and 0.5 ml of serum samples were digested with 5 ml concentrated nitric acid in a Microwave Digestion System (Berghof, Eningen, Germany) for 30 min and diluted 1:10 with distilled deionized water before analysis. Mineral levels in each sample were measured by flame AAS (AAS, Perkin-Elmer, Analyst 800, Norwalk, CT. United States) via documented and fully established processes at the 285.2, 248.3, 213.9, 324.8 nm wavelength for Mg, Fe, Zn, and Cu, respectively. The method was confirmed with specialized reference materials, and the precision was 98%. Mineral concentrations in tissues were expressed as wet weights.

The serum biotin was analyzed by HPLC (Shimadzu, Kyoto, Japan) as earlier defined, with minor modifications. The reversed-phase column used was a C18-ODS-3 column (250×4.6 mm, 5 m), and the biotin-containing chromatography fractions were dried under a stream of nitrogen before the assessment. Serum arginine concentrations were examined by high-performance liquid chromatography (HPLC, Shimadzu, Japan) as defined by Pieper and Dondlinger (Pieper and Dondlinger, 1997). Sera samples were extracted 1:1 in 35% (wt/vol) sulfosalicylic acid dihydrate. After mixing and centrifugation, the supernatant was mixed 1:1 with lithium-D buffer before analysis. The amino acid standard was obtained from Sigma-Aldrich Chemicals (St. Louis, United States).

For malondialdehyde (MDA) analyses, skin tissues were homogenized in ice-cold phosphate buffer solution for 5 min using both an ultrasonic (Vibra-Cell 130 VCX; Sonics & Materials, Inc., Newtown, CT) and a mechanic (IKA Works, Inc; Wilmington, NC) homogenizers and then centrifuged at 7,000 g for 15 min and the protein content in the supernatant was determined using nanodrop spectrophotometry (MaestroGen, Las Vegas, NV, United States). Then, an HPLC apparatus of Shimadzu UV-vis SPD-10 AVP detector, a CTO-10 AS VP column, and 30 mM KH2PO4 and methanol (82.5:17.5, v/v. pH 3.6) at a flow rate of 1.2 ml/min were used (Shimadzu, Japan). Column waste was monitored at 250 nm.

### Molecular Analysis

Dorsal skin protein levels of biotin-dependent carboxylase including ACC1, ACC2, PC, PCC, MCC, and mammalian target of rapamycin 2 (mTOR2; ser2481), p-p70S6K, p4E-BP1, vascular endothelial growth factor (VEGF), sirtuin 1 (SIRT1), matrix metalloproteinase-1 and -3 (MMP-1 and MMP-3), TNFα, NFκB, IL-6, IL-8, COX-2, Bax, Bcl-2 and caspase-3 were measured using the Western blotting technique as defined earlier. Skin tissues were pooled and homogenized at 4 C in an extraction buffer and centrifuged at 13,000×g for 20 min at 4 C. Protein samples were separated using 10% SDS-Page and transferred onto nitrocellulose membranes for 1 h prior to applying primary antibodies. The following primary antibodies were used: ACC1, ACC2, PC, PCC, MCC, p-mTORC2, p-p70S6K, p4E-BP1, VEGF, SIRT1, MMP-1, MMP-3, TNFα, NFκB, IL-6, IL-8, COX-2, Bax, Bcl-2, caspase-3, AP-1 (p-c-Jun and p-c-Fos), p-p38 MAPK and β-actin (Santa Cruz Biotechnology, CA, United States). After washing, the membranes were incubated with secondary goat anti-mouse antibodies (Santa Cruz Biotechnology) in Tris-buffered saline containing 0.05% Tween 20 for 1 h, and protein levels were measured densitometrically.

### Histological Examination

Dorsal skin samples (approximately 1×0.4 cm in size) were quickly collected at the end of the study and were fixed in a 10% formaldehyde solution, fixed in paraffin, and then cut into sections of 5 μm thickness utilizing methods known in the art. The slides were stained with hematoxylin and eosin (H&E) for routine histological examination and histological assessment of epidermal hyperplasia. The slides were also stained with Masson's trichrome and Verhoeffelastic stain to evaluate skin elasticity. For epidermal hyperplasia assessment, the epidermal thickness was photographed using an Olympus DP73 camera, and mean epidermal thickness was calculated based on 10 random site measurements on each slide using an optic microscope (Olympus BX53). Fine lines and wrinkles, two of the most important signs of photoaging, were evaluated both macro- and microscopically by examining microfold formation in the tissues stained with hematoxylin and eosin. Solar elastosis is another principal histological sign of photoaging, characterized as dermal collagen breakdown and accumulation of elastotic material, a bluish stain with the appearance of elastin fibers dermal infiltration of inflammatory cells. These changes in collagen were evaluated using Masson's trichrome stain and Verhoeff elastic stain. The intensity of inflammatory cell infiltration was expressed as the number of cells per mm².

For immunohistochemistry, collected skin tissues were fixed in 10% neutral formalin for 48 h at room temperature. Subsequently, tissue samples were embedded in paraffin and cut into sections (5 μm thick) using a microtome. The sections were then fixed onto slides. The sections were stained using rat-specific MMP-1 (Thermo Fisher Scientific, WA, United States), COX-2, IL-6, and mTOR (Abcam, Cambridge, MA, United States) antibodies with the help of an automatic immunohistochemical staining device (Ventana, Tucson, AZ, United States). We performed quantitative analyses of the immunoreactivities against MMP-1, COX-2, IL-6, and mTOR antibodies using a light microscope (Olympus BX53) equipped with a digital camera (Olympus DP73) and connected to a PC monitor. For MMP-1, COX-2, IL-6, and mTOR, the staining (0, 0%; 1, <25%; 2, 25-50%; 3, 51-75%; and 4, >75%) were scored.

### Statistical Analysis

Statistical analyses were performed using SPSS for Windows version 21.0 (IBM Corp., Armonk, NY, United States), with a one-way analysis of variance or Kruskal-Wallis test. Then Tukey post hoc test or Mann Whitney U test were used for multiple comparisons. A p value of <0.05 was considered significant.

### Results

### Biochemical Parameters

As shown in Table 1, there were no significant changes in serum glucose, liver (ALT, AST), and kidney (Urea-N, creatinine) function among groups, indicating no hepato-and nephrotoxic effects of the combination of ASI and MgB in rats exposed to UVB (p>0.05). UVB exposure caused a marked decrease in skin hydroxyproline levels and biotinidase activity by 46.1 and 42.8% compared to shaved rats (Table 1; p<0.001). Compared to the UVB group, hydroxyproline and biotinidase levels increased in all ASI+MgB treatment groups (p<0.05). The ASI+MgB-H+MgB-C group showed the greatest increase in hydroxyproline levels (65.8%) and biotinidase activity (68.2%) (p<0.05) (Table 1). Elastase activity in the dorsal skin increased from UVB irradiation by 53.3% compared to shaved rats (p<0.05), whereas ASI+MgB treatments exerted protection against the increase in dorsal skin elastase activity induced by UVB irradiation (p<0.05). The effect was more pronounced in the ASI+MgB-H group compared to the ASI+MgB-L group. However, a combination of ASI+MgB oral administration and MgB cream provided even greater preventive effects, resulting in a 30.6% decrease in elastase levels compared to the UVB group. The effects of the combination of ASI and MgB on the MDA levels, a lipid peroxidation marker, induced by UVB radiation in the skin tissue of rats also were examined. The concentration of MDA was higher in the UVB-treated group than in the no radiation groups, whereas the concentration decreased significantly in the skin of ASI and MgB-treated groups (p<0.001). The effect of the high dose of MgB combined with ASI and MgB cream was much better compared to the low doses (Table 1).

TABLE 1
Effect of inositol-stabilized arginine silicate complex (ASI) and magnesium biotinate
(MgB) on serum biochemical parameters in UV induced photoaging in rats.

| | Groups | | | | | | |
|---|---|---|---|---|---|---|---|
| | | | | | | ASI + | ASI + |
| | | | | ASI + | ASI + | MgB-L + | MgB-H + |
| Item | NC | SC | UV | MgB-L | MgB-H | MgB-C | MgB-C |
| Glucose, mg/dL | 92.57 ± | 91.00 ± | 93.86 ± | 94.00 ± | 90.57 ± | 92.14 ± | 91.43 ± |
| | 2.50 | 3.38 | 2.96 | 2.18 | 3.26 | 1.72 | 1.72 |
| BUN, mg/dL | 20.60 ± | 20.96 ± | 20.46 ± | 20.41 ± | 21.20 ± | 20.07 ± | 20.59 ± |
| | 0.85 | 0.47 | 1.40 | 1.01 | 0.94 | 1.04 | 0.94 |
| Creatinine, mg/dL | 0.11 ± | 0.12 ± | 0.13 ± | 0.11 ± | 0.11 ± | 0.12 ± | 0.12 ± |
| | 0.01 | 0.01 | 0.01 | 0.02 | 0.02 | 0.01 | 0.01 |
| TP, g/dL | 6.81 ± | 7.23 ± | 6.90 ± | 7.10 ± | 6.24 ± | 6.80 ± | 6.74 ± |
| | 0.10^{ab} | 0.08^{a} | 0.10^{ab} | 0.07^{ab} | 0.09^{b} | 0.22^{ab} | 0.37^{ab} |
| Albumin, g/dL | 3.71 ± | 3.61 ± | 3.60 ± | 3.64 ± | 3.54 ± | 3.40 ± | 3.69 ± |
| | 0.10 | 0.15 | 0.02 | 0.04 | 0.08 | 0.12 | 0.19 |
| ALT, U/L | 81.29 ± | 82.43 ± | 93.71 ± | 87.00 ± | 84.29 ± | 86.29 ± | 83.43 ± |
| | 1.48 | 2.28 | 3.20 | 2.65 | 2.83 | 3.01 | 3.13 |
| AST, U/L | 116.86 ± | 118.29 ± | 124.86 ± | 121.14 ± | 120.43 ± | 121.57 ± | 120.29 ± |
| | 5.19 | 3.27 | 5.10 | 2.94 | 3.43 | 3.31 | 5.02 |
| ALP, U/L | 168.86 ± | 167.14 ± | 133.43 ± | 147.57 ± | 157.29 ± | 146.14 ± | 156.71 ± |
| | 6.67^{a} | 5.69^{a} | 3.15^{c} | 4.09^{bc} | 2.89^{ab} | 2.96^{bc} | 3.97^{ab} |
| Hydroxyproline | 5.89 ± | 5.86 ± | 3.16 ± | 3.78 ± | 4.51 ± | 4.59 ± | 5.24 ± |
| g/100 g tissue | 0.03^{a} | 0.02^{a} | 0.03^{e} | 0.04^{b} | 0.04^{c} | 0.06^{c} | 0.06^{b} |
| Elastase pmol | 4.19 ± | 4.50 ± | 6.90 ± | 6.28 ± | 5.87 ± | 5.70 ± | 4.79 ± |
| nitroaniline/hr | 0.04^{g} | 0.05^{f} | 0.03^{a} | 0.04^{b} | 0.02^{c} | 0.03^{d} | 0.04^{e} |
| Biotinidase, U/L | 5.83 ± | 5.56 ± | 3.18 ± | 3.57 ± | 4.31 ± | 4.93 ± | 5.35 ± |
| | 0.04^{a} | 0.06^{b} | 0.07^{f} | 0.03^{e} | 0.11^{d} | 0.04^{c} | 0.03^{b} |
| MDA, nmol/mg | 2.59 ± | 2.78 ± | 7.75 ± | 5.74 ± | 4.61 ± | 4.34 ± | 3.52 ± |
| protein | 0.18^{f} | 0.14^{ef} | 0.29^{a} | 0.20^{b} | 0.28^{c} | 0.16^{cd} | 0.11^{de} |

NC: Normal Control; SC: Shaved Control; UV: Ultraviolet-Induced Photoaging; ASI: inositol-stabilized arginine silicate complex; MgB-L: Magnesium biotinate low dose; MgB-H: Magnesium biotinate high dose; MgB-C: Magnesium biotinate cream.
BUN: Blood Urea Nitrogen; TP: Total protein; ALT: Alanine aminotransferase; AST: Aspartate aminotransferase.
Data presented as mean and standard error.
^{a, b, c}Mean values within a row with unlike superscript letters were significantly different (P < 0.05).

TABLE 2
Effect of inositol-stabilized arginine silicate complex (ASI) and magnesium biotinate
(MgB) on serum minerals, biotin and arginine levels in UV induced photoaging in rats.

| | Groups | | | | | | |
|---|---|---|---|---|---|---|---|
| | | | | | | ASI + | ASI + |
| | | | | ASI + | ASI + | MgB-L + | MgB-H + |
| Item | NC | SC | UV | MgB-L | MgB-H | MgB-C | MgB-C |
| Mg, mg/dL | 2.77 ± | 2.33 ± | 1.75 ± | 3.64 ± | 4.84 ± | 3.68 ± | 4.65 ± |
| | 0.04^{c} | 0.12^{d} | 0.06^{e} | 0.15^{b} | 0.08^{a} | 0.13^{b} | 0.08^{a} |
| Fe, μg/dL | 499.00 ± | 469.00 ± | 396.86 ± | 436.71 ± | 457.29 ± | 442.57 ± | 459.71 ± |
| | 25.39^{a} | 19.35^{ab} | 19.84^{b} | 15.53^{ab} | 18.36^{ab} | 15.42^{ab} | 23.88^{ab} |
| Zn, μg/dL | 185.71 ± | 187.29 ± | 151.71 ± | 158.29 ± | 166.00 ± | 156.57 ± | 165.43 ± |
| | 3.74^{a} | 5.71^{a} | 4.83^{b} | 2.97^{b} | 2.63^{b} | 2.62^{b} | 2.53^{b} |
| Cu, μg/dl | 246.71 ± | 254.86 ± | 180.14 ± | 208.43 ± | 225.00 ± | 211.71 ± | 223.29 ± |
| | 6.67^{a} | 6.88^{ab} | 4.12^{e} | 6.91^{cd} | 5.13^{bc} | 4.73^{cd} | 5.05^{bc} |
| Si, μg/L | 229.49 ± | 227.43 ± | 158.71 ± | 529.43 ± | 532.29 ± | 532.71 ± | 534.86 ± |
| | 3.89^{b} | 9.23^{b} | 3.19^{c} | 5.49^{a} | 3.52^{a} | 5.64^{a} | 8.51^{a} |
| Biotin, | 89.32 ± | 88.70 ± | 56.46 ± | 118.84 ± | 156.94 ± | 119.62 ± | 157.78 ± |
| nmol/L | 2.41^{c} | 1.86^{c} | 3.59^{d} | 3.04^{b} | 3.00^{a} | 2.90^{b} | 4.71^{a} |
| Arginine, | 1.79 ± | 1.74 ± | 1.37 ± | 2.58 ± | 2.57 ± | 2.54 ± | 2.60 ± |
| nmol/L | 0.01^{b} | 0.02^{b} | 0.02^{c} | 0.04^{a} | 0.04^{a} | 0.04^{a} | 0.04^{a} |

NC: Normal Control; SC: Shaved Control; UV: Ultraviolet-Induced Photoaging; ASI: inositol-stabilized arginine silicate complex; MgB-L: Magnesium biotinate low dose; MgB-H: Magnesium biotinate high dose; MgB-C: Magnesium biotinate cream.
Data presented as mean and standard error.
^{a,b,c,d,e}Mean values within a row with unlike superscript letters were significantly different (P < 0.05).

### Magnesium, Iron, Zinc, Copper, Silicon Biotin and Arginine Analyses

Serum Mg, Fe, Zn, Cu, and Si levels were significantly reduced by 24.9, 15.4, 19.0, 29.3, and 30.2% in the UVB group compared with the shaved group, respectively (p<0.001; Table 2). ASI+MgB groups presented a significant increase in these parameters compared to the UVB group, particularly in the ASI+MgB-H+MgB-C group (p<0.05). Mg, Fe, Zn, Cu, and Si levels increased 165.7, 15.8, 9.0, 23.9, and 237.0%, respectively, compared to the UVB group. In addition, serum biotin and arginine concentrations were decreased by 36.8 and 23.5% in the dorsal skin following UVB irradiation (p<0.01). However, the combination of ASI and MgB treatment improved this reduction caused by UVB (p<0.05; Table 2). This effect was greater in the ASI+MgB-H+MgB-C group (2.2 mg ASI+160 μg MgB), with biotin and arginine levels increased by 179.5 and 89.8%, respectively.

### Macroscopic Appearance

Illustrative pictures of dorsal skin lesions caused by UVB exposure are shown, for example, in FIGS. 1-3 and 7-10. The intensity of fine lines and wrinkles, which is one of the most critical signs of photoaging, was increased by UVB irradiation, with the highest intensity observed in the UVB group (FIGS. 1-3 and 7-10). In addition, the dorsal skin of the rats in the UVB groups had an irregular and corrugated appearance compared to the control group. In contrast, non-irradiated rats in the normal and shaved groups showed neither wrinkles nor lesions. This not only further demonstrated UVB's skin-damaging effects but also showed that shaving had no macroscopic damage to the skin. Compared to the UVB group, the skin of rats in the ASI+MgB-H+MgB-C. ASI+MgB-L+C, and ASI+MgB-H groups appeared much smoother without visible lesions. In contrast, the dorsal skin of rats in the ASI+MgB-L group showed numerous wrinkles and mild erythema. At the end of the experiment, rats exposed to UVB with no other treatment had greater visual scores, demonstrating greater impairment when compared to control rats (p<0.001; e.g., FIGS. 1-14). However, the visual scores of the skin of the rats were significantly reduced by 37.8-76.0% in the ASI+MgB groups, an effect that was greatest in the ASI+MgB-H+MgB-C group (−76.0%) (p<0.05; e.g., FIGS. 1-14).

### Skin Elasticity

The pinch test evaluated rats' dorsal skin elasticity, and the representative pictures of skin after being pushed are presented in FIGS. 1-43. The pinch test indicated no difference between the rats' recovery times in the normal and shaved control groups (p>0.05). However, skin recovery time, associated with photoaging and a loss of skin elasticity, increased in all UVB groups. Skin recovery time was longest in the UVB group, with a time 6.8 times longer than that of the control group (p<0.05) (FIGS. 1-43). Compared with the UVB group, treatment with ASI+MgB significantly reduced recovery time by 34.8-62.8%, with the lowest duration found in the ASI+MgB-H+MgB-C group (−62.8%).

### Histological Examination

As established in H&E staining pictures (FIG. 1), Masson's trichrome (FIG. 2), and elastic fiber staining images (FIG. 3), normal and shaved rats presented almost similar appearances and showed normal skin layers, including normal epidermises covered by thin layer stratum corneum, wavy dermal-epidermal junction lines, regularly disseminated hair follicles, closely refined sequential collagen bundles, as well as well-distributed elastic fibers. Besides, no inflammatory infiltration was detected in these groups. After UVB-irradiation, rats' epidermises exhibited irregular hyperplasia with thickened stratum corneum. Thin and irregular collagen fibers were also observed in the dermis, showing prominent fractures (FIG. 1). In some instances, the elastic fibers were twisted, broken, or even irregularly accumulated, and the density was significantly reduced (FIG. 3).

Moreover, inflammatory infiltrations were visibly demonstrated throughout the dermis. However, ASI+MgB treatment orally or MgB topically (especially at a high dose with cream application) significantly alleviated these UV-induced skin damage features. The rats' skin in ASI+MgB-H+MgB-C revealed fairly complete epidermises and dermis, containing thin layer stratum corneum and well-regulated collagen and elastic fibers uniform thickness and distribution.

In Masson's trichrome staining, collagen is indicated as the blue-stained parts of the dermis that are not exposed to UVB. The collagen dispersal in the control group was regularly concentrated in the dermis, but the collagen dispersal was changed and destructed with UVB exposure. However, the administration of ASI+MgB prevented collagen fibers from UVB damage. Particularly, the high dose of ASI+MgB with the application of MgB cream presented stronger blue staining as compared to the control group (FIG. 2).

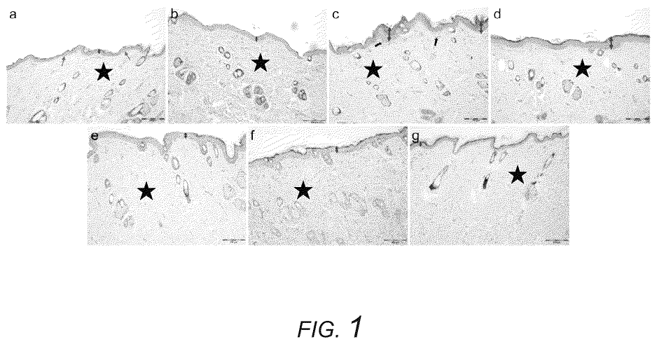

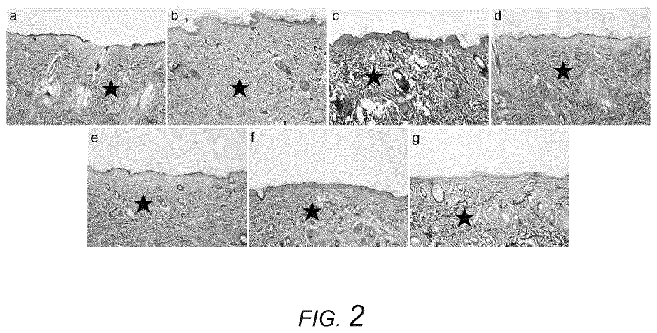

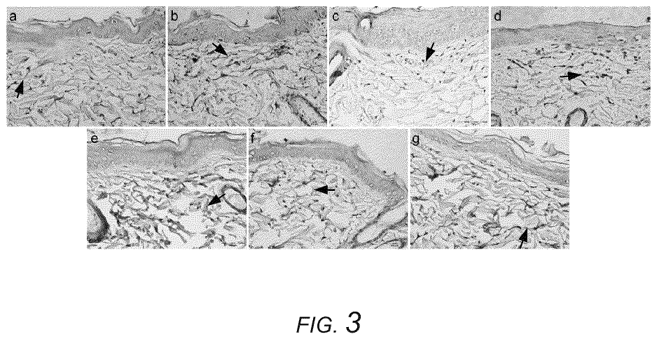

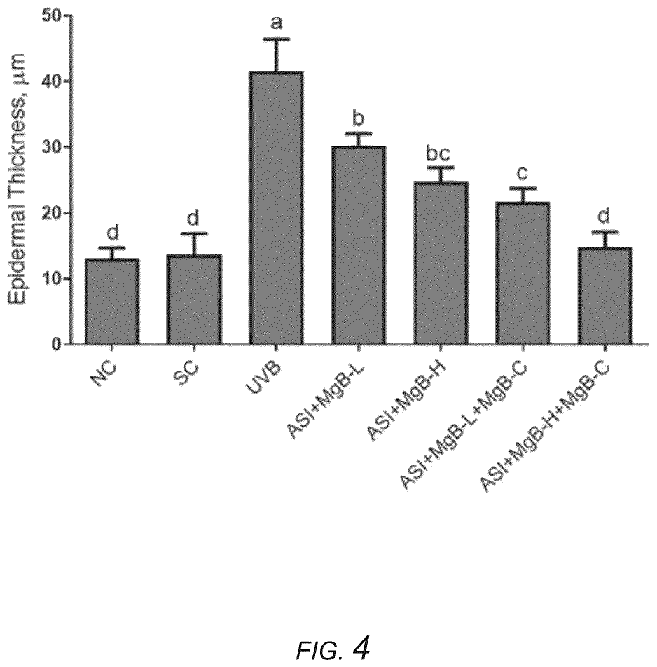

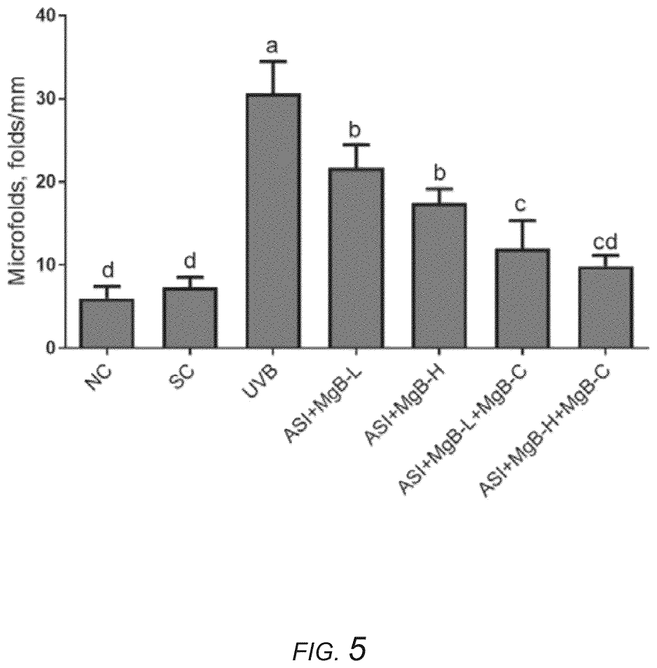

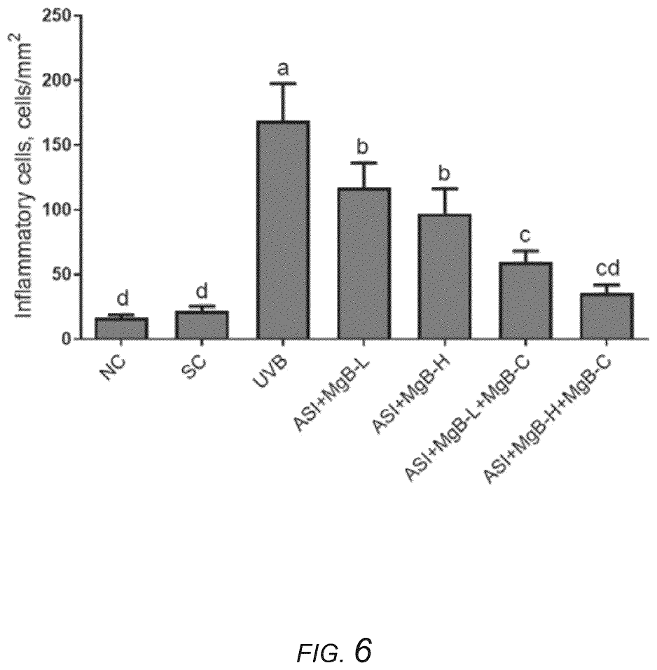

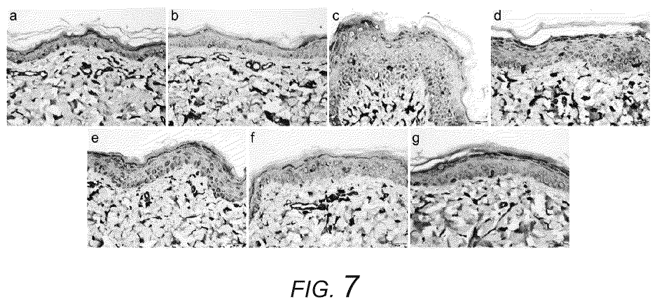

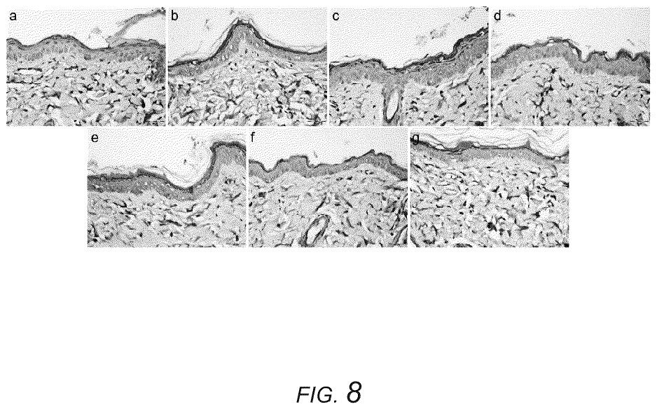

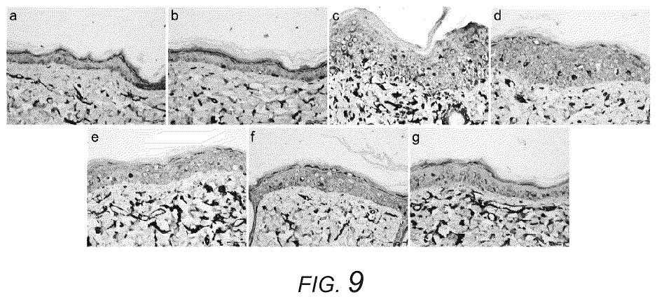

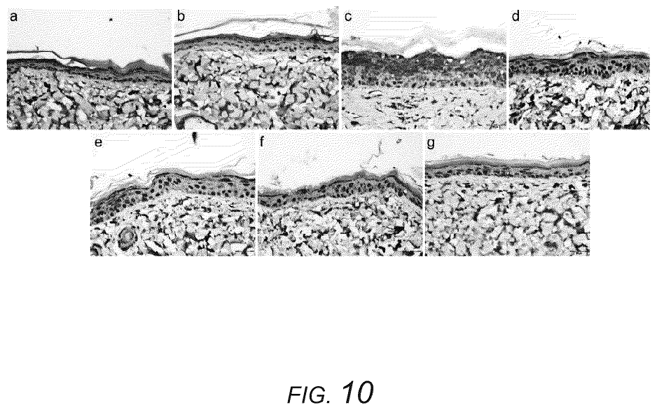

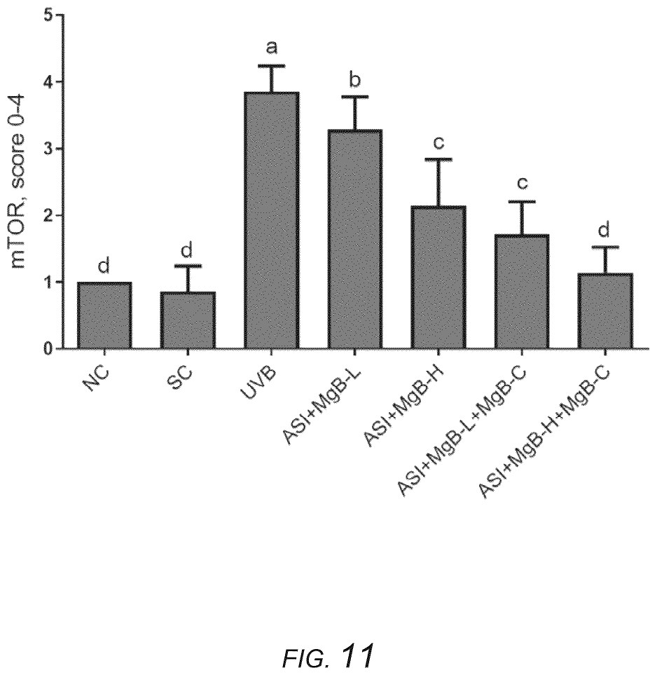

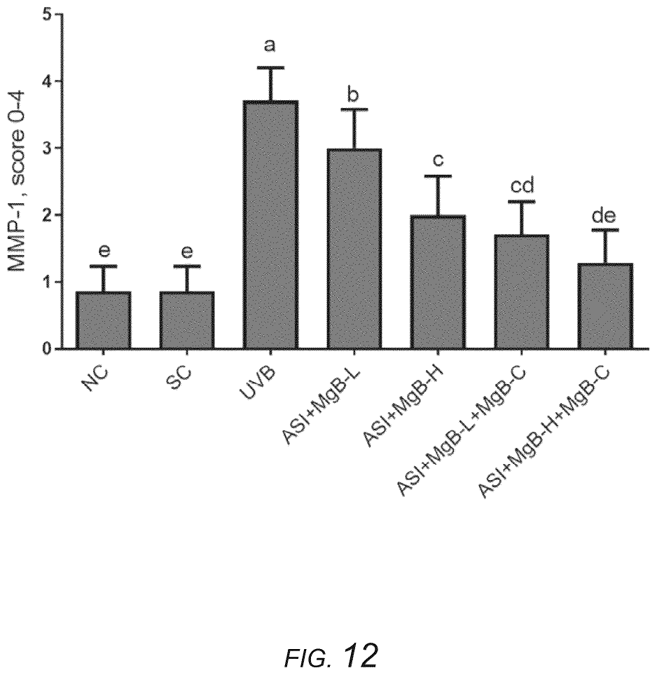

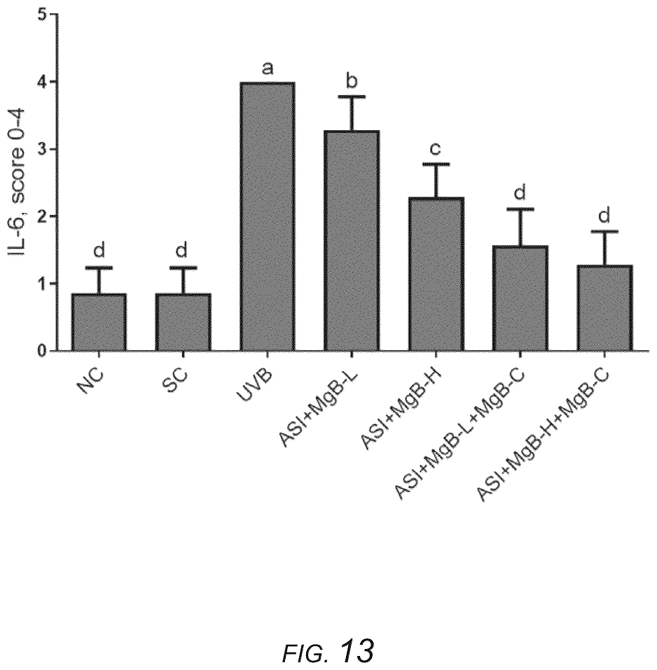

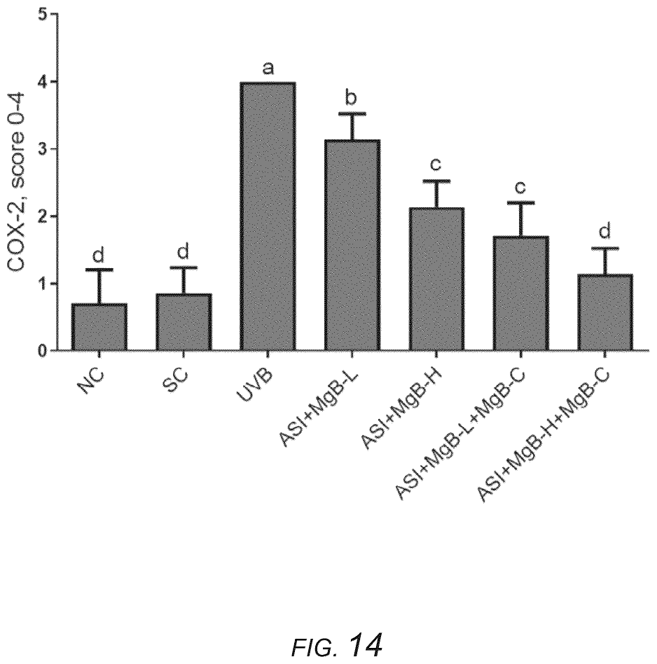

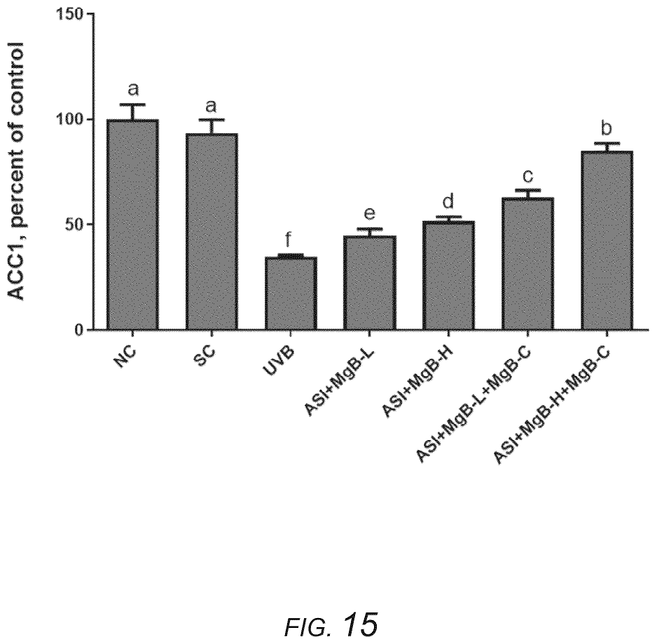

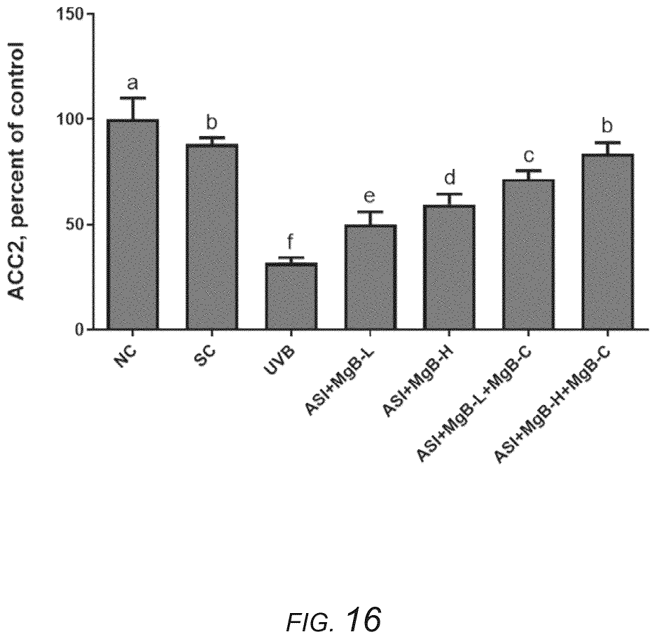

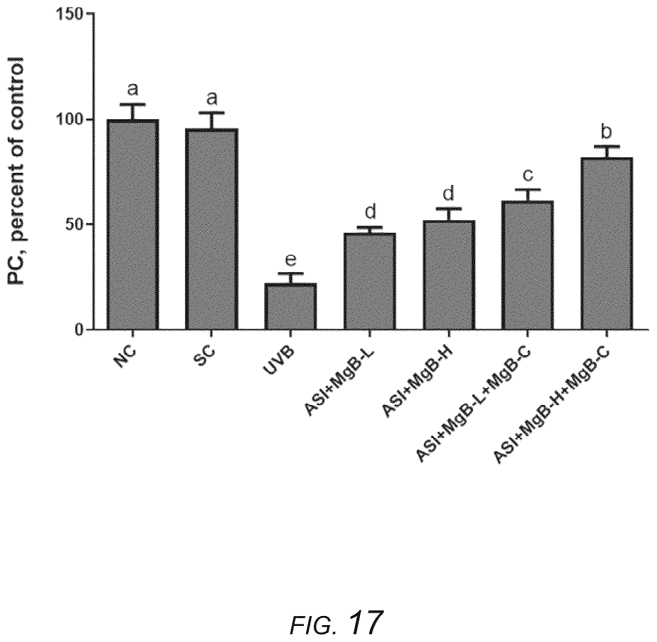

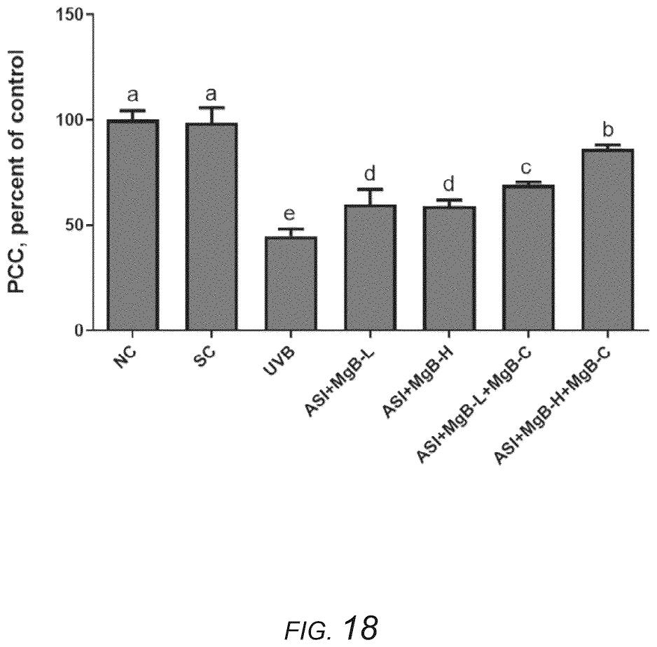

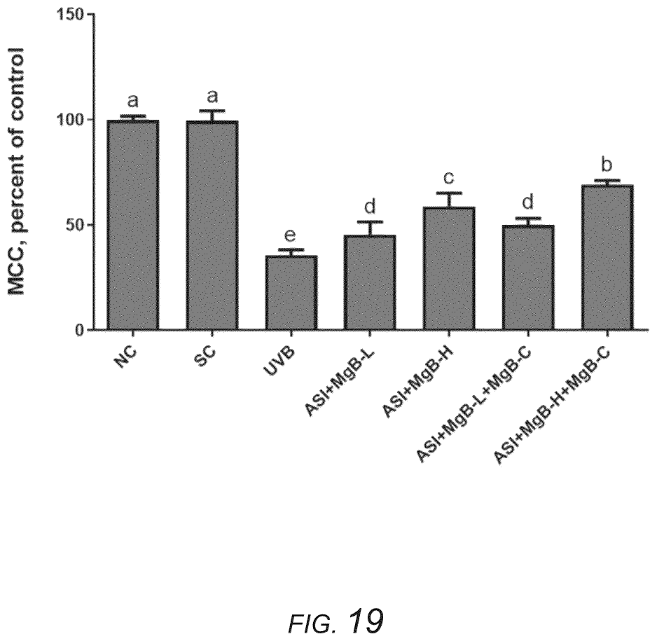

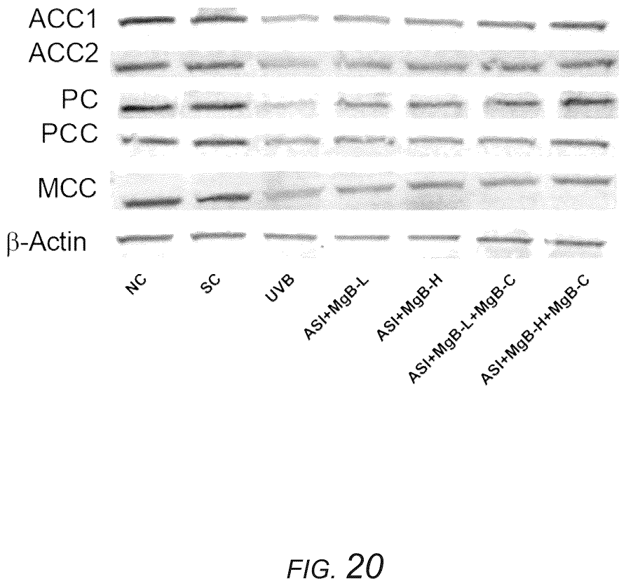

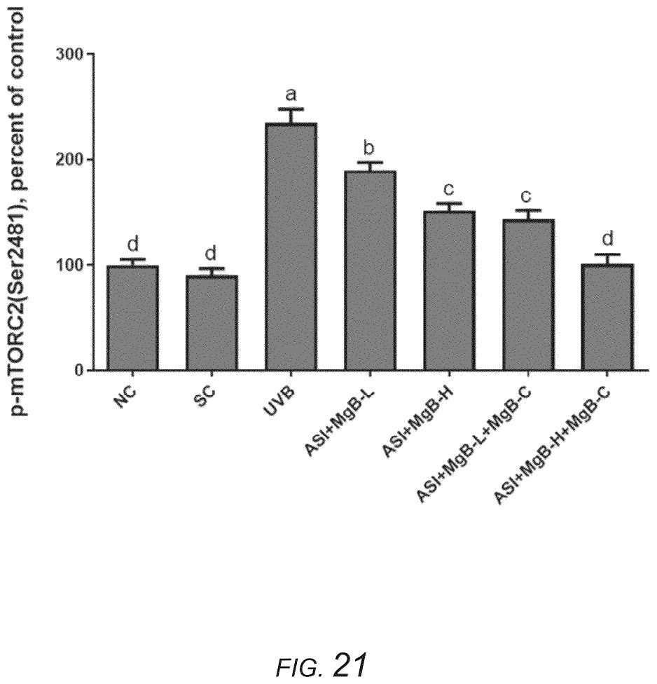

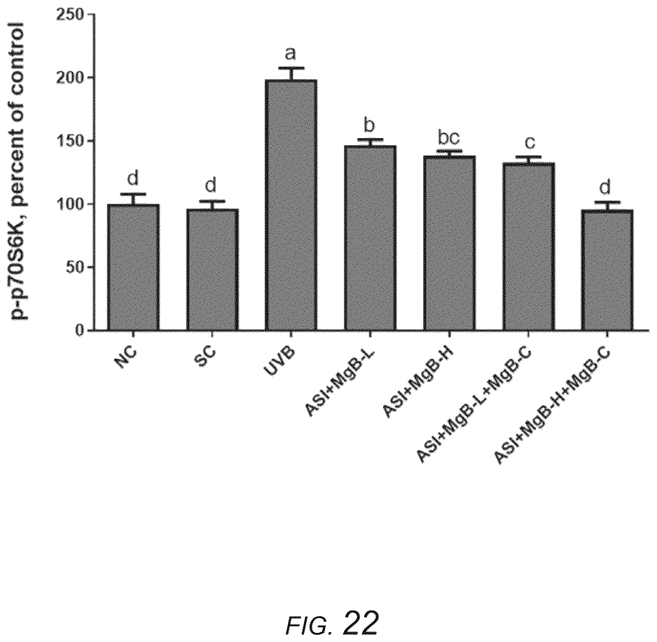

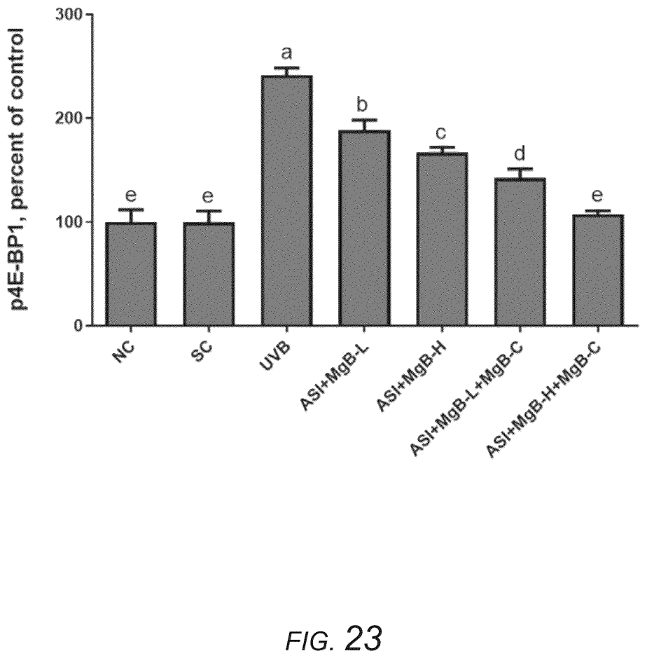

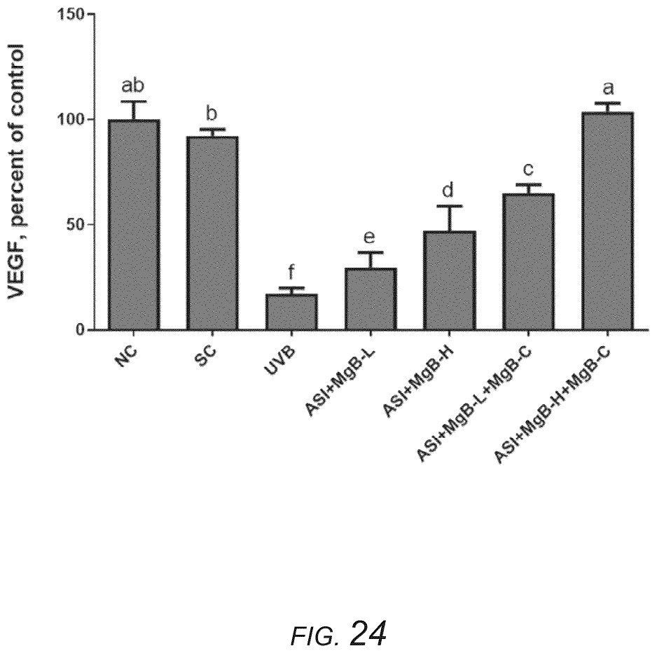

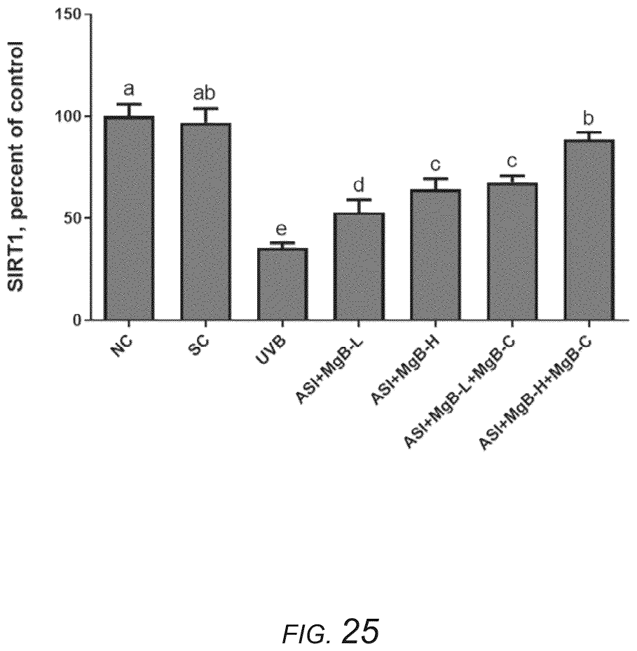

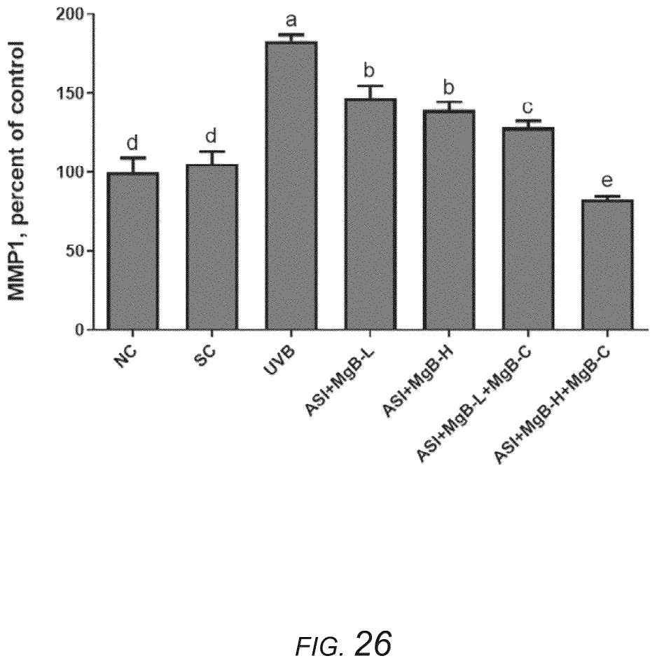

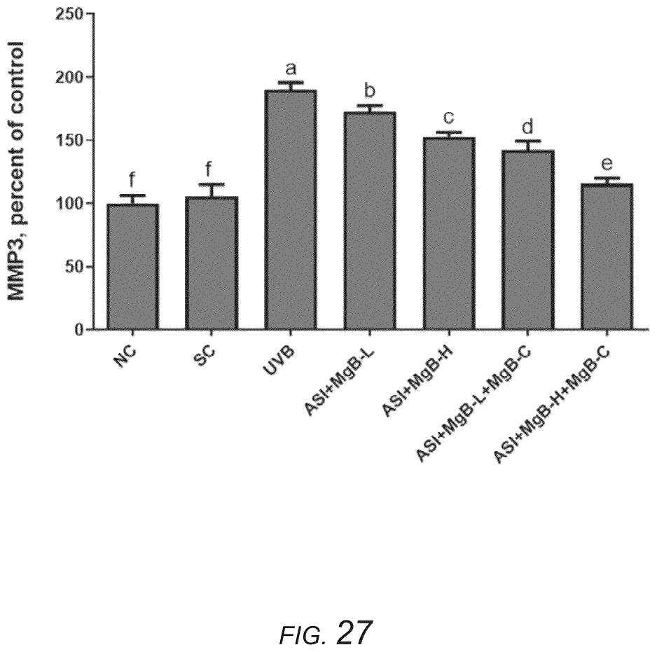

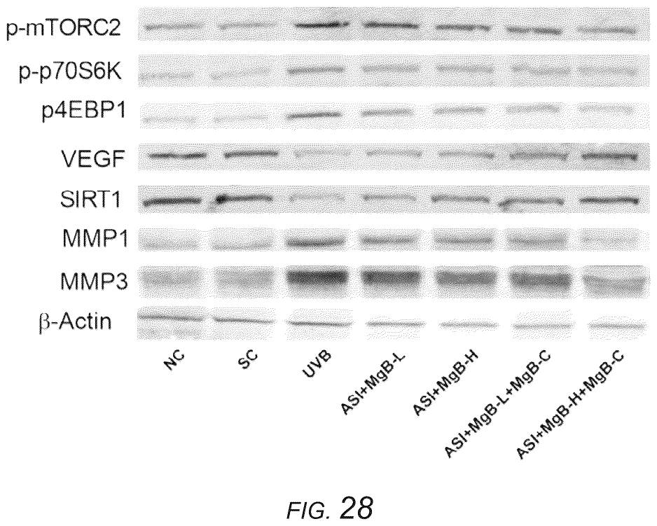

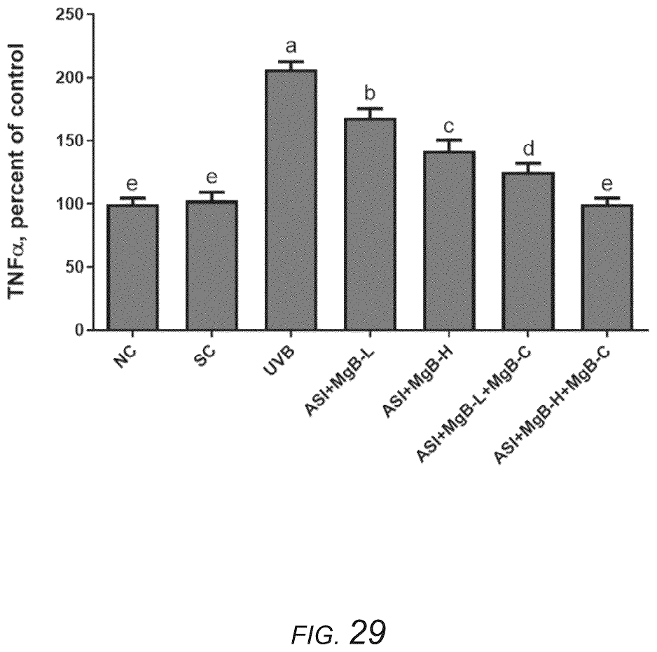

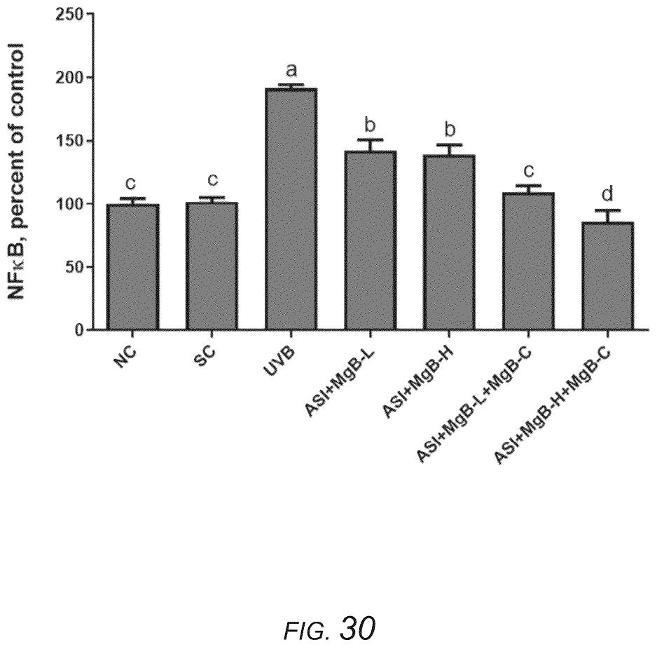

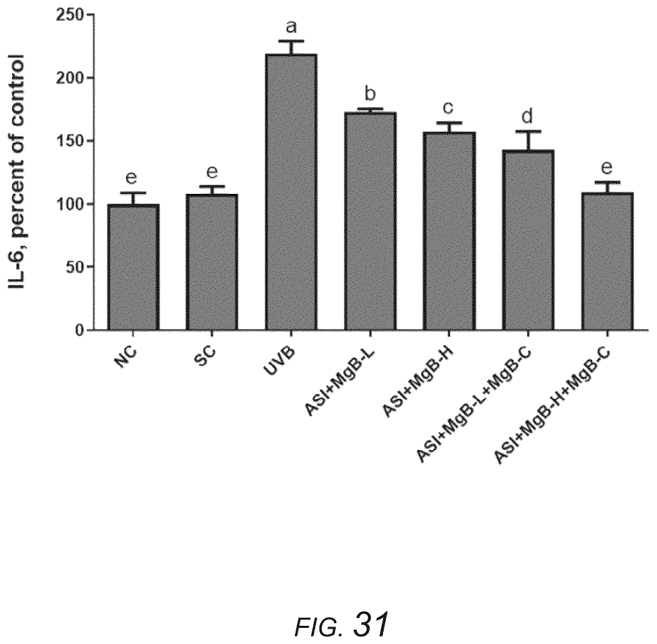

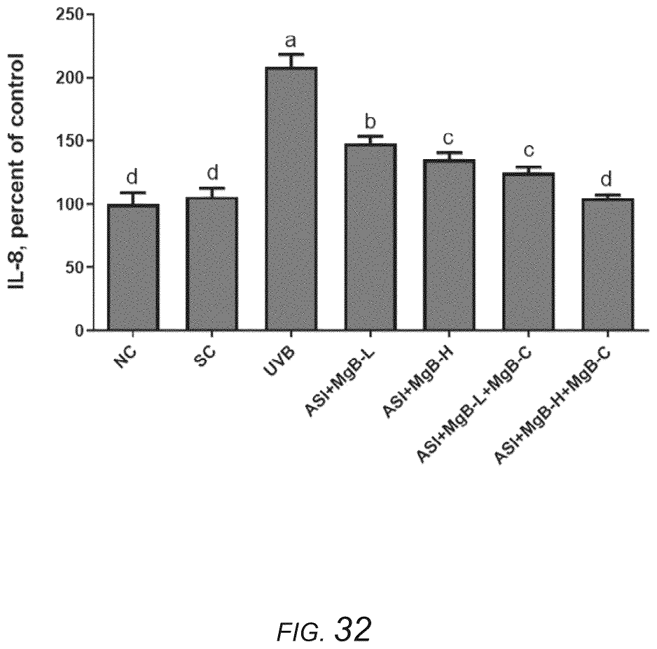

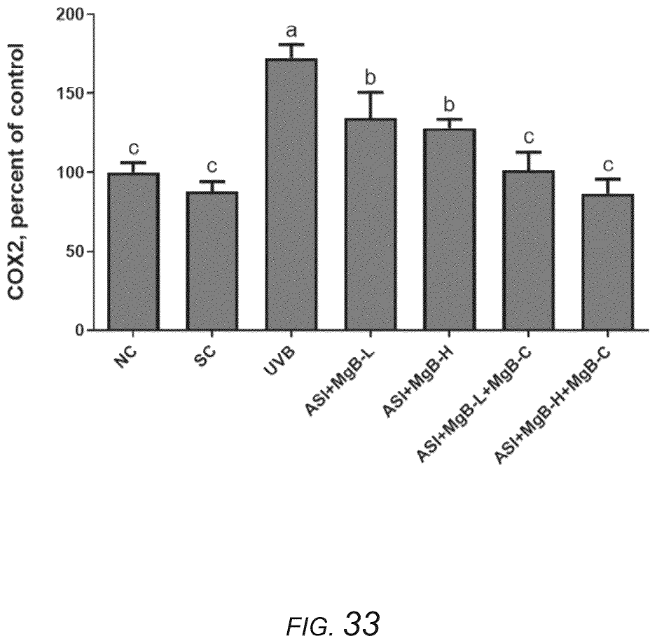

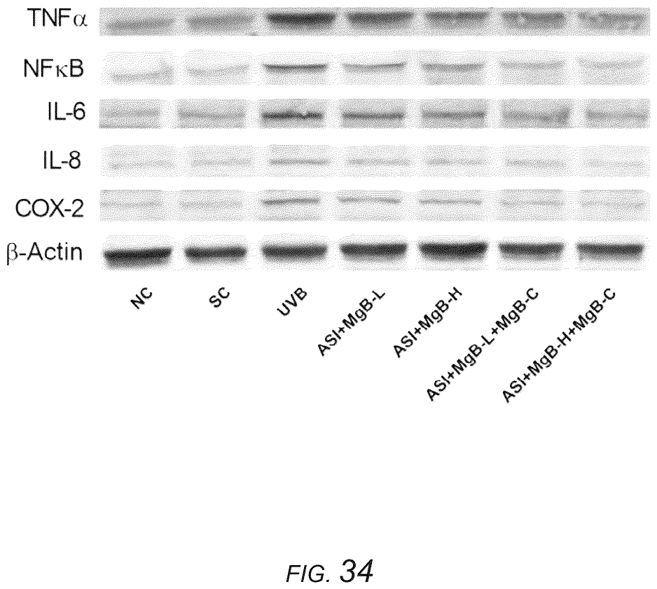

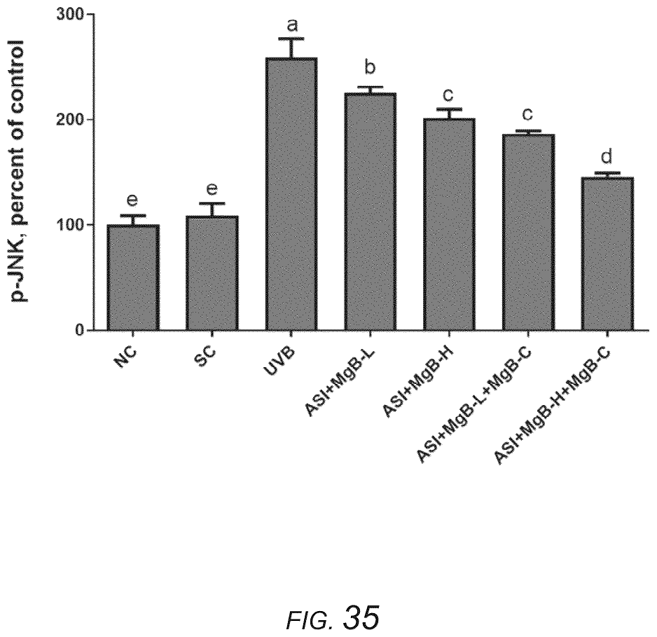

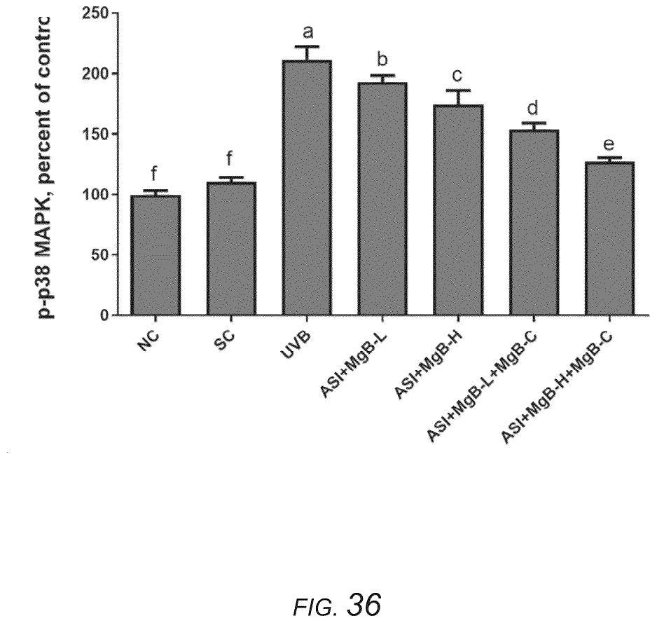

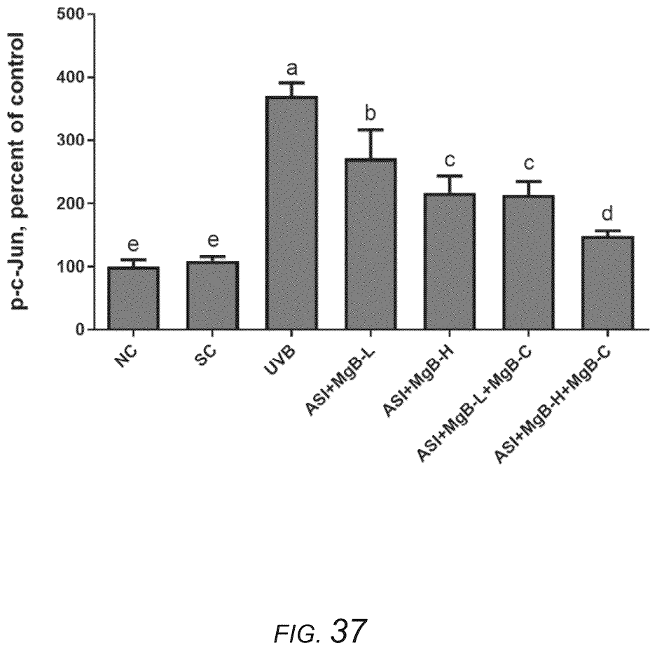

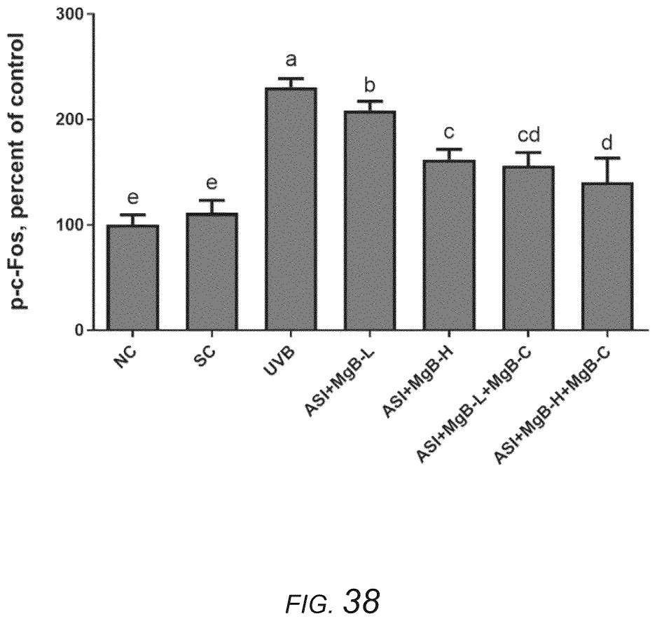

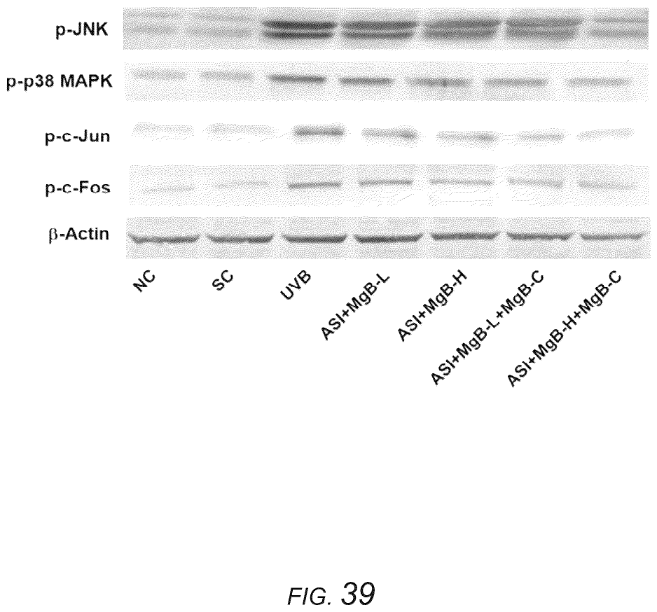

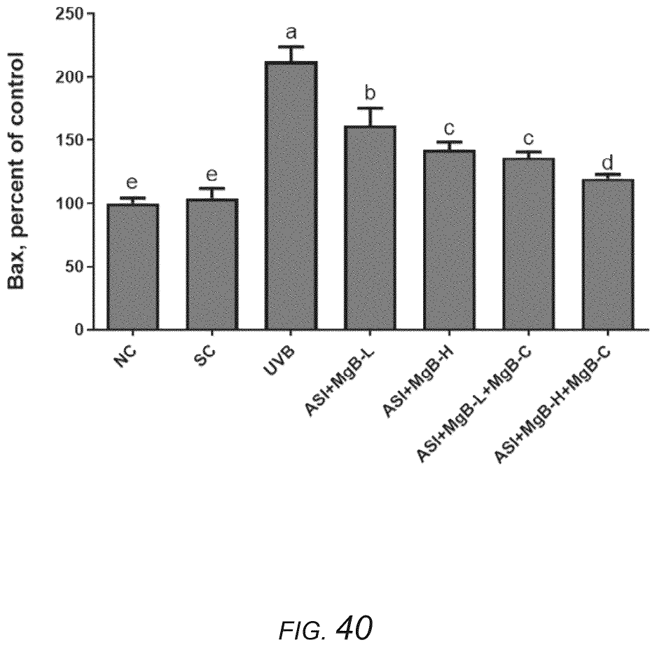

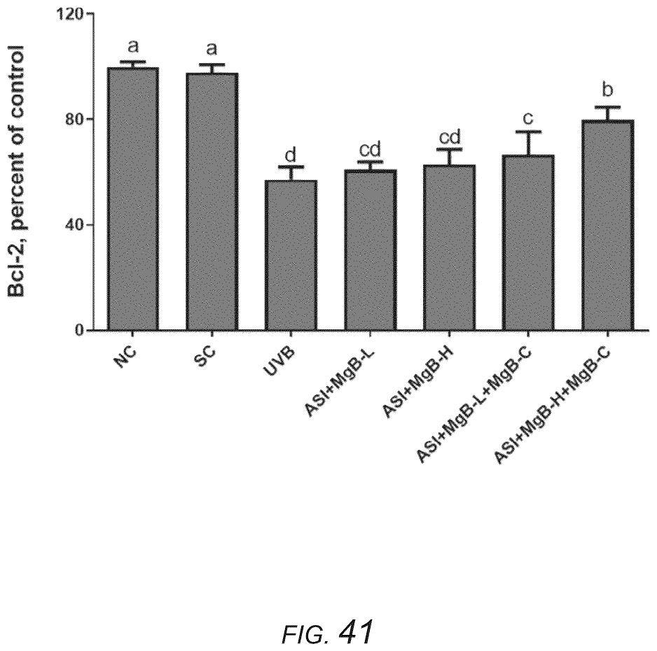

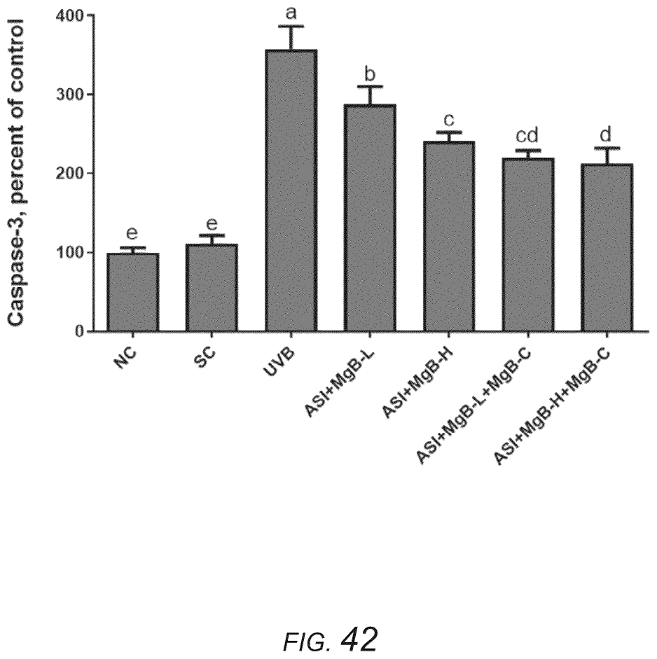

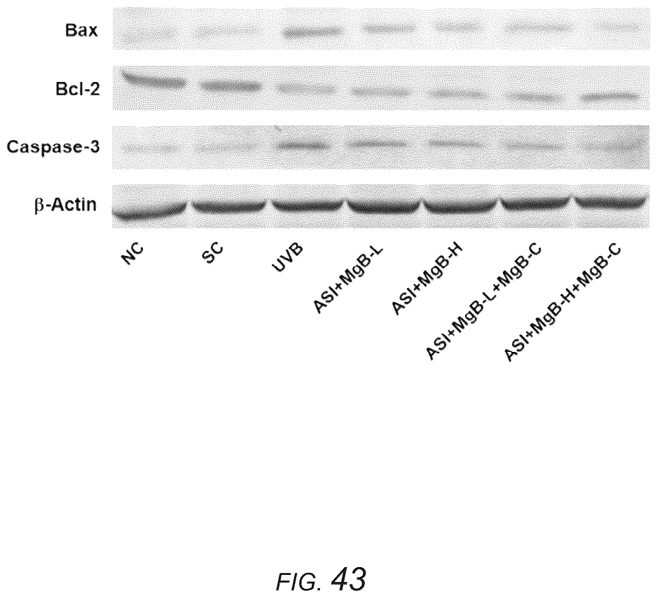

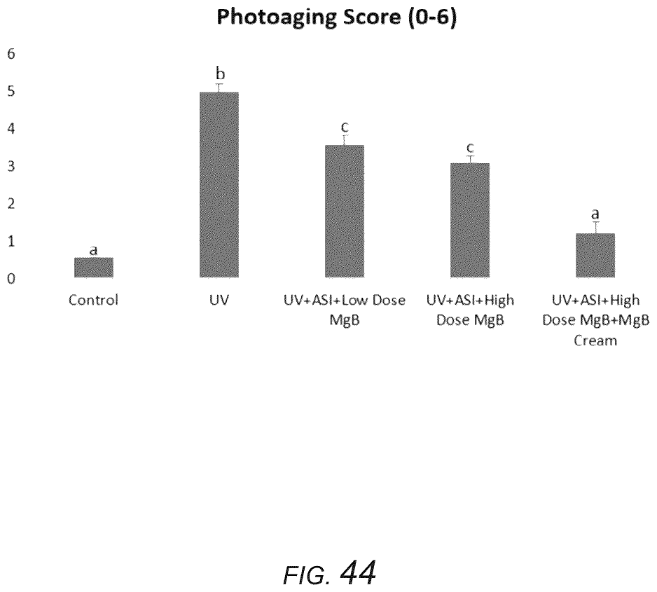

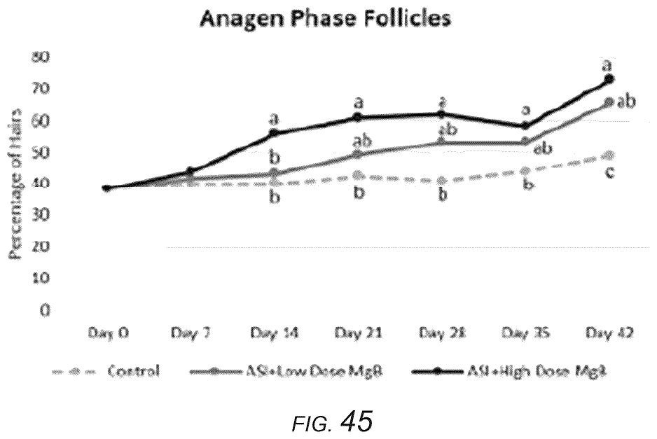

Epidermal thicknesses and microfolds of rats in the UVB group dramatically increased after chronic UVB exposure, with levels 3.0 and 4.2 times that of shaved rats, respectively (FIG. 1). However, the epidermal thickening was markedly decreased to 27.2, 40.3, 47.7, and 64.3% in the ASI+MgB-L, ASI+MgB-H, ASI+MgB-L+C, and ASI+MgB-H+C groups, respectively, compared to the UVB group (FIG. 4; p<0.05). Microfolds decreased by 29.3, 42.9, 60.9, and 67.9% in the ASI+MgB-L, ASI+MgB-H, ASI+MgB-L+C and ASI+MgB-H+C groups, respectively, compared to the UVB group (FIG. 5; p<0.05). The intensity of inflammatory cell infiltration in the skin was 7.7 times greater in the UVB irradiation group compared to shaved controls (FIG. 6; p<0.001). The administration of a combination of ASI and MgB treatment partially prevented inflammatory cell infiltration (p>0.05), particularly in the ASI+MgB-H+C group, where infiltration decreased by 79.0%.

The effect of MgB and ASI complex treatment on the expressions of mTOR, MMP-1, IL-6, and COX-2 proteins in the skin of rats was evaluated using immunohistochemistry after UVB exposure (FIGS. 7-14). The results showed that mTOR, MMP-1, IL-6, and COX-2 immunoreactivity was increased in the UVB treatment rats skin tissues compared to the control and shaved groups (FIGS. 11-14; p<0.001), whereas ASI+MgB-H treatments decreased the expression of mTOR, MMP-1, IL-6, and COX-2 in UVB treatment rats skin tissues. The highest level of decrease was detected in a high dose of MgB combined with ASI and MgB cream group.

### Protein Levels

Compared to shaved rats, dorsal skin ACC1, ACC2, PC, PCC, and MCC protein levels decreased by 63.0, 64.0, 76.8, 54.6, and 64.2, respectively, due to UVB irradiation (FIGS. 15-20: p<0.001 for all). However, a combination of ASI and MgB treatment, especially the ASI+MgB-H with cream treatment, increased ACC1, ACC2, PC, PCC, and MCC relative to those in the UVB group (p<0.05 for all). To investigate the mechanism underlying the protective effect of ASI+MgB on UVB-induced photoaging, dorsal skin levels of VEGF, SIRT1, mTORC2 (ser2481), p-p70S6K, p4E-BP1, MMP-1, and MMP-3 were also analyzed by Western blot (FIGS. 21-28). The results showed that UV irradiation-induced a decrease in protein levels of VEGF and SIRT1 by 81.4 and 63.4% and an increase in p-mTORC2 (ser2481), p-p70S6K, p4E-BP1, MMP-1, and MMP-3 protein levels by 160.4, 106.6, 141.6, 73.7 and 80.9% as compared to shaved rats, respectively. However, ASI+MgB treatment caused a significant increase in protein levels of VEGF and SIRT1 and a decrease in mTORC2 (ser2481), p-p70S6K, p4E-BP1, MMP-1, and MMP-3 protein levels in the dorsal skin of rats exposed to UVB. In addition, the UVB irradiation caused higher levels of inflammatory factors, including TNF-α, NFκB, IL-6, IL-8, and COX-2 protein levels in rats' dorsal skin (p<0.01; FIGS. 29-34). However, ASI+MgB treatment, especially in the high dose with MgB cream application, markedly inhibited their levels (p<0.05).

In order to determine the mechanism of action of the combination of ASI and MgB, we examined the mitogen-activated protein kinases (MAPKs) and AP-1 signaling pathways known to be associated with MMP expression. MAPK signaling factors stimulate activator protein-1 (AP-1) expression, which is a transcription factor and consists of c-Jun and c-Fos subunits. For this purpose, two major components of phosphorylated c-jun N-terminal kinase (p-JNK) were measured: p-p38, and AP-1 levels by western blot analysis. As seen in FIGS. 35-39, levels of p-JNK and p-p38 were increased by UVB exposure compared to control and shaved groups (p<0.0001). ASI+MgB treatments downregulated p-JNK and p-p38 levels in rats exposed to UVB. Their levels are particularly reduced by ASI+MgB combined with MgB cream than other groups (p<0.0001). Similarly, UVB irradiation significantly activated the skin tissue AP-1 (p-c-Jun and p-c-Fos) level in rats (p<0.0001). However, ASI+MgB treatments, particularly with MgB cream application, blocked the activation of the AP-1 in UVB-irradiated rats (FIGS. 35-39, p<0.0001).

As shown in FIGS. 40-43, UVB exposure significantly increased the expressions of Bax and caspase-3 and decreased the Bcl-2 levels compared to the control and shaved groups (p<0.001). However, ASI+MgB treatments, especially in the MgB high dose with MgB cream application, markedly reversed their levels (p<0.01).

As understood by the skilled artisan, photoaging is a skin condition resulting from the chronic and cumulative effects of long-term exposure to UV radiation and is characterized by epidermal thickening, accumulation of abnormal elastin-containing material, deep wrinkles, and abnormal pigmentation. Thus, the protective effects of ASI and MgB (a newly produced and highly absorbed biotin salt) were studied following oral administration, with and without MgB topically, on preventing UVB-induced skin photoaging in rats. At the macroscopic level, the skin of rats exposed to UV were found to have a wrinkled and sagging appearance and reduced skin elasticity. At the histopathological level, degraded collagen and elastic fibers were observed. Also, increases in skin thickness, microfolds, and inflammatory cells were shown. However, oral ASI+MgB treatment, with and without MgB topical application, alleviated these skin damages by regulating biotin-dependent carboxylases, mTOR pathways, matrix metalloproteinase, and inflammatory factors. In addition, ASI+MgB treatment increased serum arginine, biotin, Mg, Fe, Zn, Cu, and Si levels, particularly the high doses of ASI and MgB combined with topical MgB cream. No known previous studies have examined the protective effects of ASI and MgB treatment against UVB-induced skin damage in rats.

Consistent with previous reports, we showed that UVB exposure induced a reduction in the concentration of hydroxyproline and activity of biotinidase and increased elastase activity, an enzyme that breaks down elastin, in the dorsal skin. However, as a result of treatment with ASI+MgB, hydroxyproline concentration and biotinidase activity improved, while elastase activity was significantly reduced.

The excessive reactive oxygen production (ROS) during UV radiation-induced skin damage causes an imbalance between prooxidant production and antioxidant defense, lipid peroxidation, DNA damage, activation and expression of proteins, and infiltration of the inflammatory cells. MDA is an important end-product of lipid peroxidation and acts as an indicator of the presence of ROS. Therefore, the MDA levels were measured in the dorsal skin samples. In the present study, treatment with ASI+MgB and MgB cream has been shown to reduce MDA formation in the skin tissue of rats. This indicates that the antioxidant mechanism of MgB and ASI may involve the upregulation of endogenous antioxidants, thus preventing lipid peroxidation.

In the current study, UVB irradiation decreased the skin activities of the biotin-dependent carboxylases (ACC1, ACC2, PC, PCC, and MCC). These carboxylases participate in different metabolic pathways, including fatty acid synthesis, metabolism of amino acids, cholesterol, and odd-chain fatty acids, gluconeogenesis, and tricarboxylic acid anaplerosis (Riveron-Negrete and Fernandez-Mejia, 2017). However, ASI+MgB administration prevented the decrease of skin ACC1, ACC2, PC, PC, PCC, and MCC levels in rats exposed to UVB.

The mTOR signaling pathway is essential for cell growth, cell proliferation, angiogenesis, protein translation, and apoptosis. Exposure to UVB is a significant environmental risk factor for skin damage, characterized by abnormal activation of Akt/mTOR. Similarly, numerous studies have shown that the mTOR pathway involved in protein synthesis and cell division is upregulated by UVB in the skin. In the present study, ASI+MgB treatment suppressed the UVB-induced mTOR pathway in the dorsal skin of rats. This is the first study to demonstrate the effects of ASI+MgB on mTOR/p70S6K signaling in UVB irradiation skin.

Studies have reported that matrix metalloproteinases (MMPs) are the primary enzyme responsible for protein and collagen degradation in the skin and are stimulated by UVB irradiation. In the present study, MMP-1 and MMP-3 levels in rats that received ASI+MgB, particularly high dose ASI+MgB with MgB cream, were lower than rats exposed to UVB irradiation. This decrease in MMP-1 and MMP-3 can be attributed to the inhibition of collagen degradation enzymes in animals receiving ASI+MgB.

It is believed that UVB induces NF-KB activation through the skin's cell surface receptors, resulting in overexpression of proinflammatory cytokines including TNF-α, IL-1, IL-6, NFKB, and AP-1. In the present study, ASI+MgB treatment inhibited the overproduction of TNF-α, NFKB, IL-6, IL-8, and COX-2 caused by UVB, as well as suppressed the UVB-induced dermal inflammatory infiltrates. This effect was particularly strong with the use of MgB in high doses and MgB cream combined with ASI.

DNA damage or ROS caused by UVB often triggers certain signaling pathways, such as MAPKs, which in turn regulate a nuclear transcription factor AP-1, known to play a role in the proliferation and survival of cells. Phosphorylated MAPKs lead to increased expression of c-Jun and c-Fos and then activate AP-1. Activated AP-1 stimulates MMP expression and degrades collagen. It is believed that activation of MAPKs such as p-JNK and p-p38 MAPK is tightly correlated with inflammation and the development of skin damage through increased expression 5 of COX-2. Moreover, AP-1 is believed to play a key role as a transcription factor involved in UVB-induced COX-2 expression in many systems. In the present study. ASI+MgB treatment significantly suppressed p-JNK, p-38, c-Jun, and c-Fos in UVB-irradiated skin tissues. These results suggest that ASI+MgB treatment suppresses the levels of MMPs, and proinflammatory cytokines by regulating the AP-1 and MAPK pathways.

UV radiation induces apoptosis through imbalanced Bcl-2 proteins family and activated caspases. The Bcl-2 protein family plays a critical step in pro-apoptotic and anti-apoptotic effects by regulating the permeability of the mitochondrial membrane. Caspases may block the cell cycle, label apoptotic cells, breakdown structural proteins in the cytoskeleton, and inactivate DNA repair enzymes, leading to apoptosis. The levels of Bax and caspase-3 were significantly increased in the UVB-irradiated skin tissues compared to the control and shaved groups, whereas ASI+MgB treatment, particularly the administration of ASI+MgB in high dose with ASI+MgB cream application, significantly reversed the increase of Bax and caspase-3 in UVB-irradiated skin tissues. Compared with the control group, the UVB exposure decreased Bcl-2, while the ASI+MgB treatments prevented this UVB-induced trend. The above results concluded that ASI+MgB treatments, ASI+MgB in high dose with ASI+MgB cream could modulate the levels of Bax, Bcl-2, and caspase-3 proteins in the skin of rats receiving UVB irradiation. However, there are no previous studies to compare these results regarding the effects of the combination of ASI and MgB treatments on apoptosis in rats.

The results described herein established that the combination of ASI complex and MgB, particularly with ASI, high-dose MgB, and MgB cream, reduced UV-induced skin photoaging by anti-inflammatory processes. This combination regulates MMPs, mTOR, pathways, and inflammatory markers, including TNF-α, NFKB, IL-6, IL-8, COX-2 (may be associated with suppression of JNK, p38 MAPK, and AP-1) and apoptosis.

### Example 2

The effects of administering a composition comprising an active ingredient mixture of inositol-stabilized arginine silicate complex (ASI) and magnesium biotinate (MgB) on the prevention of skin damage from chronic UV exposure in rats was studied.

### Method

Thirty-five female Sprague-Dawley rats (8-weeks old) were randomized into one of the following groups; 1: Control (no treatment), 2: UV (exposed to UV radiation with no other treatment), 3: UV+ASI+Low Dose MgB (140 mg human equivalent dose (HED) of ASI and 1.5 mg HED of MgB), 4: UV+ASI+High Dose MgB (140 mg HED of ASI and 10 mg HED of MgB), 5: UV+ASI+High Dose MgB (140 mg HED of ASI and 10 mg HED of MgB)+MgB cream (10 mg HED). Rat diets included egg whites to standardize absorbable biotin levels. Rats had a dorsal area of hair shaved throughout the study and were exposed to UV lamps five times per week. Each day rats were dosed with study product by oral gavage and/or cream two hours before UV exposure for ten weeks.

### Results

Rats exposed to UV radiation with no other treatment showed significant signs of skin damage. Skin appearance score improved with all treatments compared to UV exposure alone (p<0.05). This parameter improved the most in group 5 compared to all treatment groups (p<0.05), with scores similar to control, while scores did not differ between groups 3 and 4 (FIG. 44). Skin elasticity evaluation showed similar results. Inflammatory cell levels in the skin were lower and collagen content was higher in all treatment groups compared to UV exposure alone (p<0.05). In group 5, inflammatory cells were no different than control levels, and collagen content was the highest compared to all treatment groups (p<0.05).

These results establish that treatment with ASI and MgB strongly protects against the skin damage that results from chronic UV exposure. The addition of a 10 mg MgB cream further increases the ability of the treatment system to improve skin appearance, elasticity, inflammation, and overall health.

### Example 3

The effects of administering a composition comprising an active ingredient mixture of inositol-stabilized arginine silicate complex (ASI) and magnesium biotinate (MgB) on hair and nail growth in rats was studied.

### Method

Twenty-one female Sprague-Dawley rats (8-weeks old) were randomized into one of the following groups; 1: Control (no treatment), 2: ASI+Low Dose MgB (140 mg human equivalent dose (HED) of ASI and 1.5 mg HED of MgB), 3: ASI+High Dose MgB (140 mg HED of ASI and 10 mg HED of MgB). Study product was dosed daily by oral gavage for six weeks. Rat diets included egg whites to standardize absorbable biotin levels. At baseline, hair was shaved from the dorsal area of the rats and hair growth was assessed on days 7, 14, 21, 28, 35, and 42. Hair growth measurements included hair density (per square cm), and percentage of hair follicles in the anagen (growing phase) and telogen (resting phase) phases of the hair cycle. Mean nail growth was measured on days 14, 28, and 42. Blood samples were collected on day 42.

### Results

Visual observations revealed greater hair growth in both treatment groups compared to control, however the effect was more pronounced in group 3. Compared to control, the percentage of hair follicles in the anagen phase was significantly greater in group 3 from day 14 onward (FIG. 45). Compared to control, hair density was significantly greater in group 3 from day 21 onward. These parameters improved in group 2 compared to control on day 42. Blood levels of vascular endothelial growth factor, a protein associated with hair growth, were higher in both treatment groups compared to control, with levels greatest in group 3 (p<0.05). Mean nail growth data showed faster daily growth in group 3 compared to other groups (p<0.05).

### Example 4

The effects of administering a composition comprising an active ingredient mixture of inositol-stabilized arginine silicate complex (ASI) and magnesium biotinate (MgB) on hair density, anagen ratio, telogen ratio, and nail growth in rats was studied.

### Method and Results

Twenty-eight female Sprague-Dawley rats (8 weeks old) were randomized into one of the following groups: (i) group (control), shaved; (ii) group (ASI), shaved+ASI (4.14 mg/rat/day); (iii) group (ASI+MgB I), shaved+ASI (4.14 mg/rat/day)+MgB (48.7 μg/rat/day); and (iv) group (ASI+MgB II), shaved+ASI (4.14 mg/rat/day)+MgB (325 μg/rat/day). On day 42, compared with the control group, while hair density (p<0.05, p<0.01, and p<0.0001, respectively) and anagen ratio (p<0.01, p<0.01, and p<0.001) increased in the ASI, ASI+MgB I, and ASI+MgB II groups, telogen ratio decreased (p<0.01, p<0.01, and p<0.001, respectively). In the molecular analysis, VEGF, HGF, and KGF-2 increased in the ASI (p<0.01, p<0.01, and p<0.05, respectively), ASI+MgB I (p<0.0001 for all), and ASI+MgB II (p<0.0001 for all) groups when compared to the control group. FGF-2 (p<0.01) and IGF-1 (p<0.001) were found to be increased in the ASI+MgB I and ASI+MgB II groups. SIRT-1 and β-catenin increased in the ASI (p<0.05 and p<0.01), ASI+MgB I (p<0.001 for both), and ASI+MgB II (p<0.0001 for both) groups. Wnt-1 increased in the ASI+MgB I (p<0.001) and ASI+MgB II (p<0.0001) groups.

### Discussion

One of the study findings is that hair growth was better in the ASI+MgB groups than in the ASI alone groups. It was observed that the addition of biotin to ASI increased hair density and anagen ratio and decreased telogen ratio. It was determined that increased hair density and anagen ratio and decreased telogen ratio effects increased even more, when the dose of biotin was increased. This study also showed that SIRT-1 was increased in all groups. However, the more significant increase in the high-dose magnesium biotinate group suggests that SIRT-1 may be more sensitive to magnesium biotinate. Macroscopically significant nail growth could be achieved only in the group receiving 10 mg MgB combined with ASI in this study. It was determined that MgB contributed to nail growth at high doses.

In conclusion, the combination of ASI and MgB could promote hair growth by regulating IGF-1, FGF, KGF, HGF, VEGF, SIRT-1, Wnt, and β-catenin signal pathways. ASI alone did not affect nail growth, whereas the MgB combination was effective using a higher dose of biotin.

### Example 5

The effects of administering a composition comprising an active ingredient mixture of inositol-stabilized arginine silicate complex (ASI) and magnesium biotinate (MgB) on the appearance of hair, skin, and nails in healthy women was studied.

### Materials and Methods

In a randomized, double-blind study, 90 healthy female subjects with self-reported thinning hair who met Savin/Ludwig Scale criteria I-2 to II-1 by physician evaluation were randomized to one of three groups for 12 weeks (n=30/group): (ASI+MgB I), ASI (146.5 mg/subject/day, which corresponds to about 10 mg of silicon)+MgB (11.7 mg/subject/day, which corresponds to about 10 mg of biotin); (ASI+MgB II), ASI (146.5 mg/subject/day, which corresponds to about 10 mg of silicon)+MgB (3.5 mg/subject/day; which corresponds to about 3 mg of biotin); or Placebo (PL). Hair quality and thickness were measured by the TrichoScan HD testing system and skin parameters (facial wrinkles, fine lines, skin texture, skin color evenness, skin elasticity) were measured by the Antera 3D™ System and the Cutometer™ Dual MPA 580 system.

### Results

There was a significant increase in hair thickness measured by change in % vellus hair and % terminal hair and in the ratio of % vellus to terminal hair in ASI+MgB I compared to PL at Week 3, maintained throughout the study (p=0.029). ASI+MgB I had a significant decrease in facial wrinkles (12 Weeks) measured by a change in maximal wrinkle depth vs. PL (p=0.031). After 12 weeks compared to baseline ASI+MgB I significantly improved facial wrinkle Maximum Depth, Indentation Index and Score, facial fine lines Indentation Index and Score, and facial texture Maximum Height, Roughness and Score (p<0.05), no change in PL. There were no changes for skin elasticity between groups. For some hair and skin parameters, ASI+MgB II showed improvements less than ASI+MgB I but that approached significance (p<0.1). All groups improved in subjective nail endpoints vs. baseline with no significant differences observed between groups. No adverse events reported.

### Conclusion

ASI+MgB I significantly increased hair thickness and reduced facial wrinkle depth compared to placebo and performed better than the ASI+MgB II in most parameters.

### Example 6

The effects of orally administering a composition comprising an active ingredient mixture of inositol-stabilized arginine silicate complex (ASI) and magnesium biotinate (MgB) conjointly with a hair serum comprising an active ingredient mixture comprising ASI and MgB on the appearance of hair, skin, and nails in women was studied.

### Methods

Twelve (12) women were given a free supply of capsules and hair serum to use for 3 months. The capsules and hair serum each comprised an active ingredient mixture comprising ASI and MgB. Each month, subjects received online survey questionnaires with questions focusing on hair, skin, and nail health. After completion of each survey questionnaire, subjects were compensated with a $25 gift card.

### Results

The following results reflect participants' responses after using the combination of capsules and hair serum for three months. Hair surveys showed that 92% of women reported an improvement in overall hair volume, 82% of women reported an improvement in hair thickness, and 75% of women reported an improvement in hair shine. Nail surveys showed that 83% of women reported an improvement in nail strength and 83% of women reported an improvement in nail growth rate. Skin surveys showed that 75% of women reported an improvement in skin smoothness, 75% of women reported an improvement in overall skin health, 83% of women reported an improvement in how satisfied they were with how healthy their facial skin looked, and 83% of women were less bothered by how noticeable the lines on their face looked.

### Conclusions

These data establish that the combination of capsules and hair serum, each comprising an active ingredient mixture comprising ASI and MgB, improves various aspects of hair, skin, and nail health and appearance in women.

### Example 7

The effect of administering a composition comprising an active ingredient mixture comprising inositol-stabilized arginine silicate complex (ASI) and magnesium biotinate (MgB) on the appearance of skin and hair on healthy women and, optionally, healthy men is studied.

### Method

At minimum, 86 healthy women and minimally 15 healthy men between the ages of 21-65 will be recruited for a six month randomized, double-blind clinical study. Baseline measurements for each of the parameters described in Table 3 (below) will be performed for each subject. After the measurements are taken, the subjects will be divided into two groups: group 1 (minimum of 43 subjects including a minimum of 15 men) and group 2 (minimally 43 women); the women will participate in a randomized, double-blind, placebo-controlled parallel arm study. The men will participate in an open-label intervention arm embedded in the larger randomized trial with women. The subjects in group 1 will be given a three month supply of capsules containing an active ingredient mixture of ASI (146.5 mg/subject/day which contains about 10 mg of Si) and MgB (11.7 mg/subject/day which contains about 10 mg of biotin) and will be instructed to take one capsule daily. The subjects in group 2 will be given a 3-month supply of capsules containing a placebo and will be instructed to take one capsule daily. After three months, all female subjects are measured for the appropriate parameters described in Table 3 (below); men will only be evaluated for the hair parameters. All the subjects will then be given an additional three month supply of capsules equivalent to their initial supply. After another three months, all female subjects will be measured again for the appropriate parameters described in Table 3 (below); male subjects will again only be evaluated for hair parameters.

TABLE 3
Study Design

| | | | | 6 Months |
|---|---|---|---|---|
| Claim | Methodology | Baseline | 3 Months | (Final) |
| FEMALE SUBJECTS | | | | |
| (n = min 43/grp) | | | | |
| Primary Claims-Hair | | | | |
| Increased Hair Growth | Medicam 1000- | Yes | Yes | Yes |
| | non-shaving | | | |
| | measurement | | | |
| Increase Hair Shine | Glossmeter | Yes | Yes | Yes |
| Increased Hair Thickness | Medicam 1000 | Yes | Yes | Yes |
| Before and After Photography | 3 angles-Digital | Yes | Yes | Yes |
| | Photography | | | |
| Secondary Endpoints-Skin | | | | |
| Reduces appearance of fine | Vaestro analysis of | Yes | Yes | Yes |
| lines and | canfield | | | |
| wrinkles | VISIA-CR images | | | |
| Improves Skin texture | Visioscan | Yes | Yes | Yes |
| Improves skin | Cutometer | Yes | Yes | Yes |
| elasticity/firmness | | | | |
| Improves Skin Hydration | Corneometer | Yes | Yes | Yes |
| Subjective Assessments | Questionnaire* | N/A | Yes | Yes |
| | (Hair/Skin/Nails) | | | |
| MALE SUBJECTS | | | | |
| (min 15 subjects) | | | | |
| HAIR ONLY | | | | |
| Increased Hair Growth | Medicam 1000- | Yes | Yes | Yes |
| | non-shaving | | | |
| | measurement | | | |
| Increase Hair Shine | Glossmeter | Yes | Yes | Yes |
| Increased Hair Thickness | Medicam 1000 | Yes | Yes | Yes |
| Before and After Photography | 3 angles-Digital | Yes | Yes | Yes |
| | Photography | | | |
| Subjective Assessments | Questionnaire* | N/A | | Yes |

### Results

After three months and after six months, the female subject population of group 1 and group 2 will be compared as to their hair growth, hair shine, and hair thickness. Further, after three months and after six months, the female subject population of group 1 and group 2 will be compared as to their skin texture, skin elasticity/firmness, and skin hydration, as well as any reduction in the appearance of fine lines and wrinkles. The groups will also be compared as to self-reporting in the questionnaire regarding improvement in the appearance of their skin and hair after three months and after six months. Male subjects in the open-label arm of the trial will be evaluated at 3 months and 6 months for hair-related outcomes, and will also complete the questionnaire to assess self-reported changes in hair, skin, nails, and general satisfaction with appearance.

### INCORPORATION BY REFERENCE

All publications and patents mentioned herein are hereby incorporated by reference in their entirety. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A composition comprising an active ingredient mixture comprising silicon and biotin.

2. The composition of claim 1, wherein the mass ratio of the silicon to the biotin is from 1:5 to 5:1.

3. The composition of claim 2, wherein the active ingredient mixture comprises from 5 mg to 20 mg silicon and from 1 mg to 15 mg biotin.

4. The composition of claim 1, wherein the mass ratio of the silicon to the biotin is from 10,000:1 to 5:1.

5. (canceled)

6. The composition of claim 1, wherein the active ingredient mixture further comprises magnesium.

7. The composition of claim 1, wherein the biotin is a biotin salt.

8. (canceled)

9. (canceled)

10. The composition of claim 7, wherein the biotin salt is magnesium biotinate.

11. (canceled)

12. (canceled)

13. The composition of claim 10, wherein the magnesium biotinate is magnesium D-biotinate.

14. The composition of claim 1, wherein the active ingredient mixture comprises arginine, an arginine silicate complex, a polyol, inositol, or a combination thereof.

15-17. (canceled)

18. The composition of claim 1, wherein the active ingredient mixture comprises an inositol-stabilized arginine-silicate complex (ASI).

19. The composition of claim 18, wherein the ASI has a molar ratio of arginine to silicate to inositol of about 2:2:1.

20-24. (canceled)

25. A composition formulated to deliver silicon and biotin to a subject.

26. The composition of claim 6, formulated to deliver from 5 mg to 20 mg silicon and from 1 mg to 15 mg biotin to the subject.

27. (canceled)

28. The composition of claim 1, wherein the composition is formulated for oral administration.

29. The composition of claim 28, formulated as a dispersible powder, a beverage, a gummy, a gel, a tablet, a hard capsule or a soft capsule.

30-33. (canceled)

34. The composition of claim 1, wherein the composition is formulated for extended release, controlled release, or a combination thereof.

35. The composition of claim 1, wherein the composition comprises a pharmaceutically acceptable excipient.

36. A method of improving the appearance of a subject's skin, hair, or nails comprising administering a composition of claim 1 to the subject.

37. The method of claim 36, wherein the method improves skin elasticity, skin hydration, skin texture, facial wrinkle depth, skin inflammation, hair pigmentation, hair thickness, hair density, hair volume, hair shine, hair strength, nail strength, nail growth, hair growth, or a combination thereof.

38. The method of claim 36, wherein the method improves hair growth.

39. The method of claim 38, wherein the method increases the duration of the anagen stage of hair growth.

40. A method of treating or preventing a condition in a subject comprising administering a composition of claim 1 to the subject, wherein the condition comprises hair thinning, hair loss, skin wrinkles, photodamaged skin, irregular skin pigmentation, nail brittleness, a genetic condition, menopause, aging, chronic stress, chronic inflammation, a side effect of a medication, or photodamaged skin caused by exposure to ultraviolet B radiation.

41. (canceled)

42. (canceled)

43. The method of claim 40, wherein the condition comprises a side effect of a medication.

44. The method of claim 40, wherein the condition comprises photodamaged skin caused by exposure to ultraviolet B radiation.

45. (canceled)

46. The method of claim 36, wherein the composition is administered orally to the subject.

47-51. (canceled)

52. The method of claim 36, wherein the composition is administered at least once per day.

53. The method of claim 36, wherein the subject is a human.

54. The method of claim 53, wherein the subject is female.

55. The method of claim 53, wherein the subject is male.

56. The method of claim 36, wherein a daily dosage of the active ingredient mixture comprises from 5 mg to 20 mg silicon and from 1 mg to 15 mg biotin.

57. (canceled)
